(19) United States
(12) Patent Application Publication
(10) Pub. No.: US 2026/0256694 A1
(43) Pub. Date: Sep. 3, 2026

(54) Multilamellar Vesicle Drug Formulation

(71) Applicant: Nanostar Pharmaceuticals Ltd., Vancouver (CA)

(72) Inventors: Shyh-Dar LI, Vancouver (CA); Nojoud AL FAYEZ, Vancouver (CA); Roland BOETTGER, Vancouver (CA); Sreemoyee GHOSH, Vancouver (CA); Po-Han CHAO, Vancouver (CA); Jing LI, Vancouver (CA)

(21) Appl. No.: 18/881,755

(22) PCT Filed: Jul. 7, 2023
(86) PCT No.: PCT/US2023/027062

§ 371 Date: Jan. 7, 2025

Related U.S. Application Data
(60) Provisional application No. 63/359,190, filed on Jul. 7, 2022.

(51) Int. Cl. A61K 9/127; A61K 31/138; A61K 31/4168; A61K 31/4178; A61K 31/437; A61K 31/4706; A61K 31/4709; A61K 31/4745; A61K 31/496; A61K 31/506; A61K 31/5415

(52) U.S. Cl. CPC A61K 9/127; A61K 31/138; A61K 31/4168; A61K 31/4178; A61K 31/437; A61K 31/4706; A61K 31/4709; A61K 31/4745; A61K 31/496; A61K 31/506; A61K 31/5415

(57) ABSTRACT

The present invention provides a multiple lamellar vesicle active pharmaceutical ingredient (MLV-API) drug formulation comprising MLV in aqueous basic solution wherein the MLV encapsulates the API and wherein the API comprises a weak base compound. The MLV-API is prepared without the use of extrusion or dialysis procedures and is able to achieve high encapsulation efficiency of 90% as well as high drug to lipid ratio of over 15% by weight percentage. The present invention also provides a method as well as a kit for preparation of the MLV-API drug formulation.

### FIELD OF THE INVENTION

The present invention relates to composition and method of preparation of a multilamellar vesicle drug formulation with high encapsulation efficiency that can be made without extrusion or dialysis procedures.

### BACKGROUND OF THE INVENTION

Most medicines are only available in solid dosage forms such as tablets and capsules, which are difficult for children to swallow^{1,2}. Extemporaneous compounding of those adult dosage forms is required for pediatric use. This typically involves dividing an adult dosage form into smaller doses, which is then ground up into powder and mixed with food or liquid, such as water, milk or juice. Unfortunately, this common practice is associated with poor medication adherence^{3,4}. First, the dosage is often not accurate, posing safety and efficacy concerns for many drugs that have a narrow therapeutic window. Second, many drugs exhibit an unpleasant taste that cannot be effectively masked by food or flavored liquids. Often, children spit out or refuse to take the medicine, leading to failure of therapy⁵.

Tang et al.⁶ were the first to show that liposomal small unilamellar vesicles (SUVs) were an effective platform for preparing child-friendly drug formulations. They encapsulated a highly bitter drug, mefloquine (Mef), in the aqueous core of SUV liposomes to disperse the drug in a solution form for easy swallowing and also to prevent direct contact of Mef molecules with the taste buds in the mouth, leading to complete masking of the taste. Upon oral ingestion, SUV liposomes effectively released the payload in the acidic gastric fluid as well as in the bile salt containing intestinal fluid, resulting in slightly increased oral absorption compared to the standard suspension formulation. However, manufacturing the SUV liposomes was tedious, time consuming, expensive, and challenging for scale-up. The method involved hydrating a thin lipid film composed of cholesterol and a phospholipid (DSPC) with aqueous ammonium sulfate to prepare liposomal multilamellar vesicles (MLVs), followed by membrane extrusion to reduce the particle size and lamellarity, yielding liposomal SUVs. Then, the SUVs were dialyzed against 10% sucrose to create a transmembrane gradient. Finally, the drug was mixed with the SUVs and incubated to facilitate active loading into the SUV core⁶. The membrane extrusion and dialysis procedures are particularly challenging for scale-up. In addition, it was long thought that drugs could not be actively loaded into MLVs due to the limited aqueous space between the lipid bilayers. However, the present invention is the first to successfully perform an active loading method for MLV liposomes with high encapsulation efficiency and D/L ratio. Therefore, there is a need for an alternative liposomal technology based on MLV that does not require membrane extrusion and dialysis to enable substantially easier preparation and scale-up manufacturing.

### SUMMARY OF THE INVENTION

The present invention provides a multilamellar vesicle active pharmaceutical ingredient (MLV-API) drug formulation comprising MLV in an aqueous basic solution and an API wherein the API comprises a weak base drug; wherein the MLV encapsulates the API; wherein the drug to lipid ratio of the API encapsulated MLV is about between about 15% to about 25% by weight percentage; and wherein the pH within the MLV is lower than the pH of the aqueous basic solution at the exterior of the MLV.

The present invention also provides a method of preparation of a MLV-API drug formulation comprising the steps of a. form MLV by hydrating organic components with an acidic solution; d. add an aqueous basic solution to the MLV formed from step a; e. dissolve an API in water to form API solution; f. mix the API solution from step e with the aqueous basic solution containing MLV of step d to result in an active loading solution in which a pH gradient is established between inside and outside of the MLV to facilitate active loading of the API; f. incubate the active loading solution of step f to cause active loading of the API into the MLV wherein the API comprises a weak base; wherein the organic components comprise one or more lipids capable of forming the MLV; and wherein the method of preparation does not include the use of dialysis or extrusion procedures.

The present invention also provides a MLV-API drug formulation preparation kit comprising an API, an aqueous basic solution and lyophilized MLV wherein the API comprises a weak base drug in powder form or any form capable of being ground into powder form; wherein the three components are stored separately; and wherein mixing the three components results in active loading of the API into the MLV without requiring any additional ingredients and without extrusion and dialysis procedures to form an MLV-API drug formulation.

### BRIEF DESCRIPTION OF THE DRAWINGS

FIG. 1 illustrates encapsulation efficiency of chloroquine (CQ) into various embodiments of the MLV drug formulation of the present invention each with different drug-lipid ratios (D/L).

FIG. 2A illustrates encapsulation efficiency of Nile Blue (NB) into the various embodiments of the MLV drug formulation of the present invention at different drug-lipid ratios (D/L). FIG. 2B illustrates encapsulation efficiency of HCQ into the MLV drug formulation of the present invention at different drug-lipid ratios (D/L).

FIG. 3 illustrates characterization of empty and MLV-CQ drug formulation of the present invention.

FIG. 4 illustrates characterization of MLV-NB and MLV-HCQ drug formulation of the present invention.

FIG. 5A illustrates cryo-TEM images of empty MLV of the MLV drug formulation of the present invention and FIG. 5B illustrates cryo-TEM images of MLV-CQ of the MLV drug formulation of the present invention at a drug to lipid ratio (D/L) of 20 wt %.

FIG. 6 illustrates schematic diagram of an embodiment of the MLV drug formulation kit of the present invention and an embodiment of the procedure of formulation compounding of the present invention. The kit includes a bottle of lyophilized MLVs (A) and a bottle of aqueous Na₂CO₃ (B). Step 1: Reconstituting MLVs with a pre-determined volume of water; Step 2: Adjusting pH to 8-10 using a pre-determined volume of aqueous NaCO3; Step 3: Compounding a tablet/capsule into powder and adding it into MLVs with thorough mixing; Step 4: Incubating the mixture at room temperature for 1 h before use.

FIG. 7A illustrates CQ encapsulation efficiency in lyophilized MLV drug formulation of the present invention stored at room temperature for up to 8 weeks. FIG. 7B illustrates CQ encapsulation efficiency in MLV-CQ drug formulation of the present invention stored at 4° C. for up to 4 weeks.

FIG. 8 illustrates HCQ encapsulation efficiency into an embodiment of the MLV drug formulation of the present invention by compounding a HCQ adult tablet into a liquid preparation using an embodiment of the MLV drug formulation kit of the present invention at a range of D/L ratios.

FIG. 9 illustrates in vitro drug release profiles of an embodiment of the MLV-CQ (20%, w/w) drug formulation of the present invention in simulated saliva (SS), simulated gastric fluid (SGF), and simulated intestinal fluid (SIF) supplemented with 3 mM sodium taurocholate.

FIG. 10 illustrates pharmacokinetic profiles of an embodiment of the MLV-CQ drug formulation of the present invention and free CQ administrated orally to female CD-1 mice at 10 mg CQ/kg.

FIG. 11 illustrates pharmacokinetic parameters of an embodiment of the MLV-CQ drug formulation of the present invention and CQ solution after oral administration in female CD-1 mice at 10 mg/kg.

FIG. 12 illustrates size and PDI measurements of empty MLV drug formulation of the present invention using dynamic light scattering (DLS).

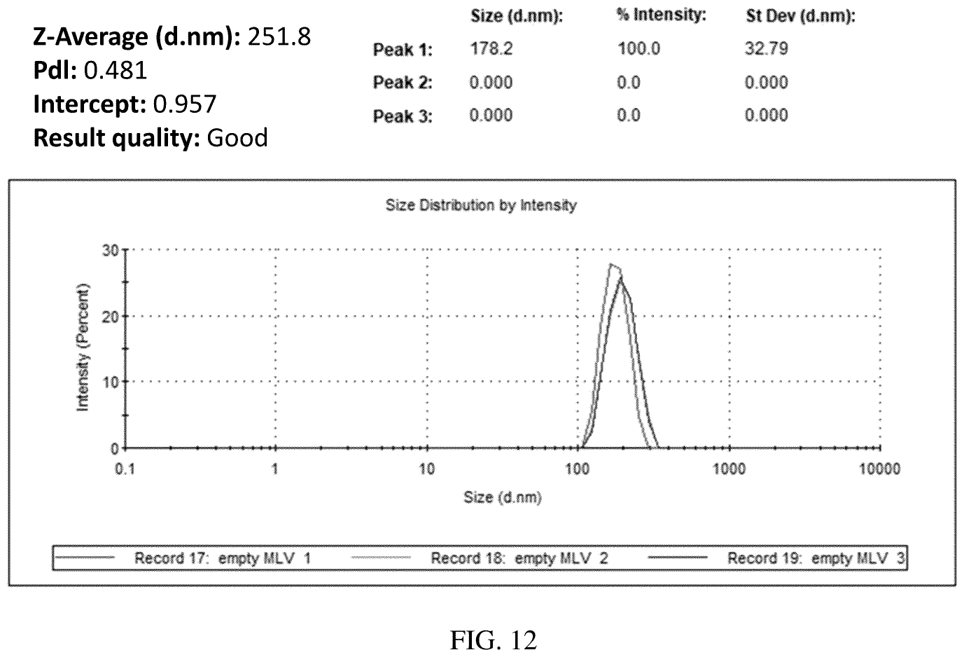

FIG. 13 illustrates size and PDI measurements of an embodiment of the MLV-CQ drug formulation of the present invention using DLS.

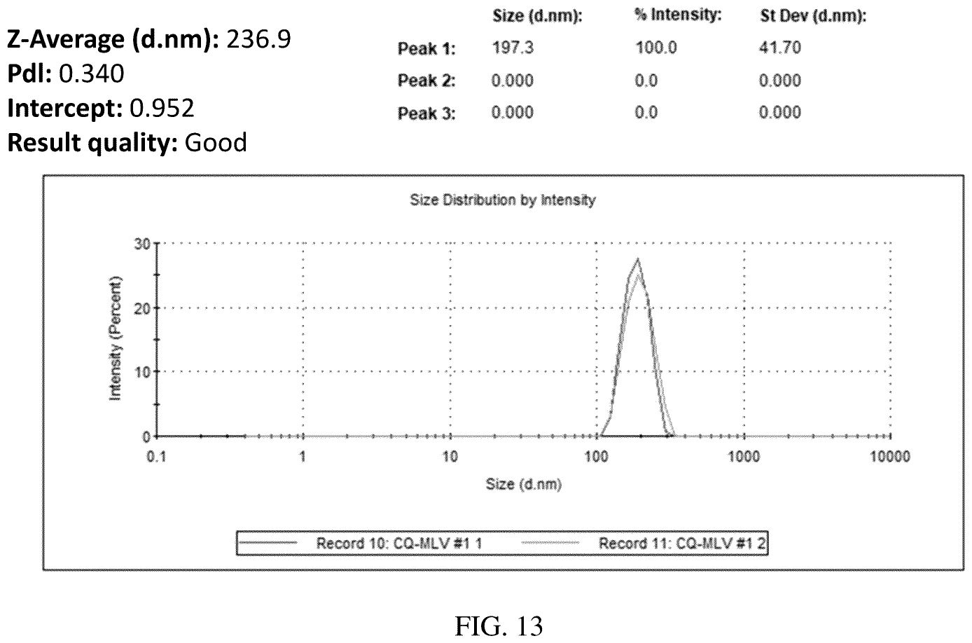

FIG. 14 illustrates size and PDI measurements of an embodiment of the MLV-HCQ drug formulation of the present invention using DLS.

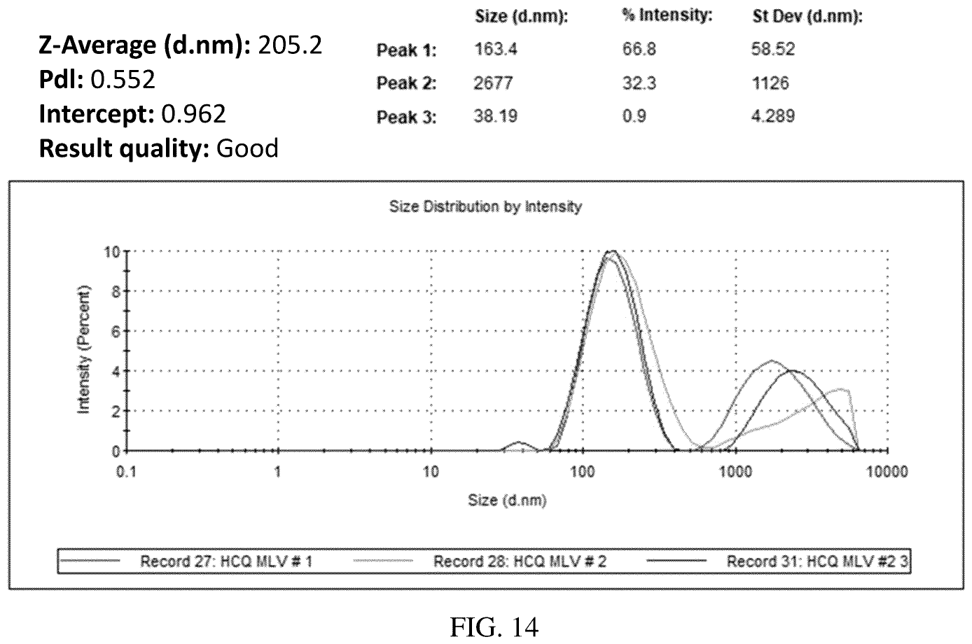

FIG. 15 illustrates size and PDI measurements of an embodiment of the MLV-NB drug formulation of the present invention using DLS.

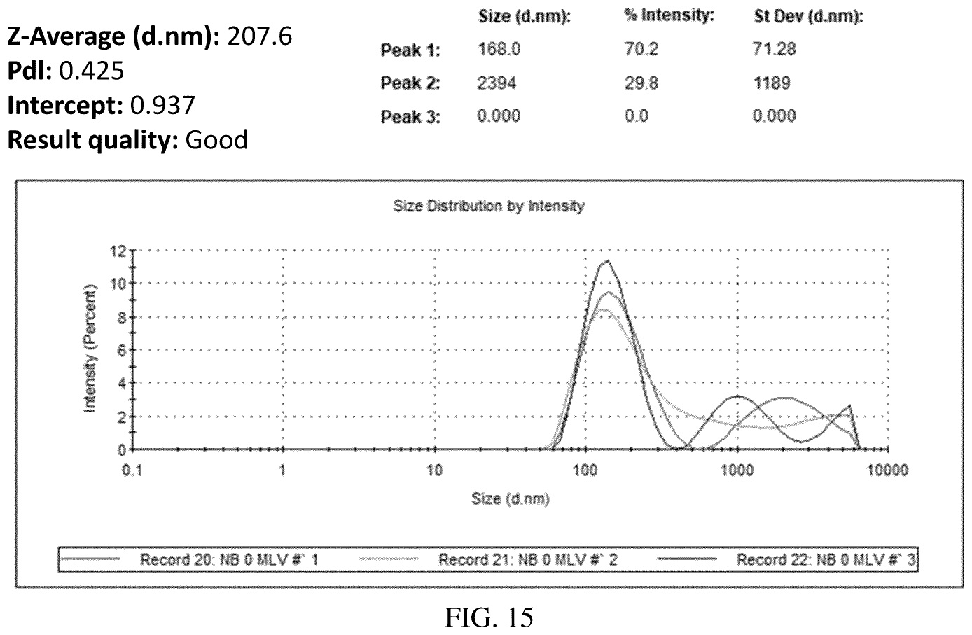

FIG. 16 illustrates clonidine retention rate (RR, %) of MLV-clonidine drug formulation of the present invention during storage at 4° C.

FIG. 17 illustrates clonidine encapsulation efficiency (EE %) of MLV-clonidine drug formulation of the present invention during storage at 4° C. as a function of time.

### DETAILED DESCRIPTION OF THE PRESENT INVENTION

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof.

“About” in the context of amount values refers to an average deviation of maximum ±20%, preferably ±10% or more preferably ±5% based on the indicated value. For example, an amount of about 30 mol % anionic lipid refers to 30 mol %±6 mol %, preferably 30 mol %±3 mol % or more preferably 30 mol %±1.5 mol % anionic lipid with respect to the total lipid/amphiphile molarity.

A “therapeutically effective amount” of a compound comprises administering an amount necessary to achieve a desired result. The exact amount required will vary from subject to subject, depending on the species, age, general condition of the subject, the severity of the disease, its mode of administration, the desired outcome, and the like. In certain embodiments of the present invention, a “therapeutically effective amount” of a compound or pharmaceutical composition is that amount effective for alleviating symptoms of, inhibiting progression or reversing of any disease disclosed herein in a subject or a biological sample (e.g., in cells). In certain embodiments, symptoms of the disease is alleviated by about 10%, about 20%, about 30%, about 40%, about 50%, about 60%, about 70%, about 80%, about 90%, about 95%, or about 99%. In certain embodiments, the disease progression is inhibited by about 10%, about 20%, about 30%, about 40%, about 50%, about 60%, about 70%, about 80%, about 90%, about 95%, or about 99%. In certain embodiments, the compound inhibits disease progression by at least about 25%, at least about 50%, at least about 75%, or at least about 90%. In certain embodiments, the disease is reversed by about 1%, about 5%, about 10%, about 20%, about 30%, about 40%, about 50%, about 60%, about 70%, about 80%, about 90%, about 95%, about 99% or any numbers and number ranges falling within these values.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

“Single lamellar vesicles” are spherical vesicles that includes one lipid bilayer membrane which defines a single closed aqueous compartment. The bilayer membrane includes two layers of lipids; an inner layer and an outer layer (leaflet). The outer layer of the lipid molecules are oriented with their hydrophilic head portions toward the external aqueous environment and their hydrophobic tails pointed downward toward the—interior of the liposome. The inner layer of the lipid lays directly beneath the outer layer, the lipids are oriented with their heads facing the aqueous interior of the liposome and their tails toward the tails of the outer layer of lipid.

“Multiple lamellar vesicles” include more than one lipid bilayer membrane, which membranes define more than one closed aqueous compartment. The membranes are concentrically arranged so that the different membranes are separated by aqueous compartments, much like an onion.

The present invention provides a multiple lamellar vesicle active pharmaceutical ingredient (MLV-API) drug formulation comprising MLV in aqueous basic solution wherein the MLV encapsulates the API and wherein the API comprises a weak base compound. In an embodiment, the API encapsulated within the MLV comprises chloroquine (CQ), CQ derivative (e.g., hydrochloroquine (HCQ)), clonidine, clonidine derivatives, imatinib, propranolol, pilocarpine, meloxicam, aripiprazole, chlorpromazine, R848, imiquimod, mefloquine and primaquine or a pharmaceutically acceptable salt thereof. In an embodiment, any embodiment of the MLV-API formulation of the present invention disclosed herein is prepared by active loading of the API into the MLV of the present invention and is done without the use of dialysis and/or extrusion procedures. In an embodiment, the MLV-API drug formulation of the present invention is prepared by active loading of the API into the MLV of the present invention and is done without the use of dialysis or extrusion wherein the encapsulation efficiency is at least about 70%, at least about 80%, at least about 90%, at least about 95%, at least about 96%, at least about 97%, at least about 98% or at least about 99% such that less than about 10%, less than about 5%, less than about 4%, less than about 3%, less than about 2% or less than about 1% of the API is present in the aqueous basic solution outside of the MVL. In an embodiment, the encapsulation efficiency of MLV-API drug formulation of the present invention is greater than about 95% when D/L weight ratio within the active loading solution is between about 5 to about 20 by weight %. In an embodiment, the encapsulation efficiency of MLV-CQ is greater than about 95% when D/L weight ratio within the active loading solution is between about 15 to about 20 by weight %. In an embodiment, the encapsulation efficiency of MLV-NB is greater than about 95% when D/L weight ratio of the active loading solution is about 5 by weight %. In an embodiment, the encapsulation efficiency of MLV-HCQ is greater than about 95% when D/L weight ratio of the active loading solution is between about 5 to about 15 by weight %.

In an embodiment, the drug to lipid (D/L) ratio of the API loaded MLV of the present invention is between 5-30 by weight percentage such as about 5, about 10, about 15, about 20, about 25 or about 30 by wt % including any numbers and number ranges falling within these values. In an embodiment, the drug to lipid (D/L) ratio of the CQ loaded MLV (MLV-CQ) of the present invention may be between about 10 to about 30 by weight percentage such as about 10, about 15, about 20, about 25 or about 30 by wt % including any numbers and number ranges falling within these values. In an embodiment, the drug to lipid (D/L) ratio of the NB loaded MLV (MLV-NB) of the present invention may be between about 10 to about 30 by weight percentage such as about 10, about 15, about 20, about 25 or about 30 by wt % including any numbers and number ranges falling within these values. In an embodiment, the drug to lipid (D/L) ratio of the HCQ loaded MLV (MLV-HCQ) of the present invention may be between about 5 to about 25 by weight percentage such as about 5, about 10, about 15, about 20, about 25 by wt % including any numbers and number ranges falling within these values.

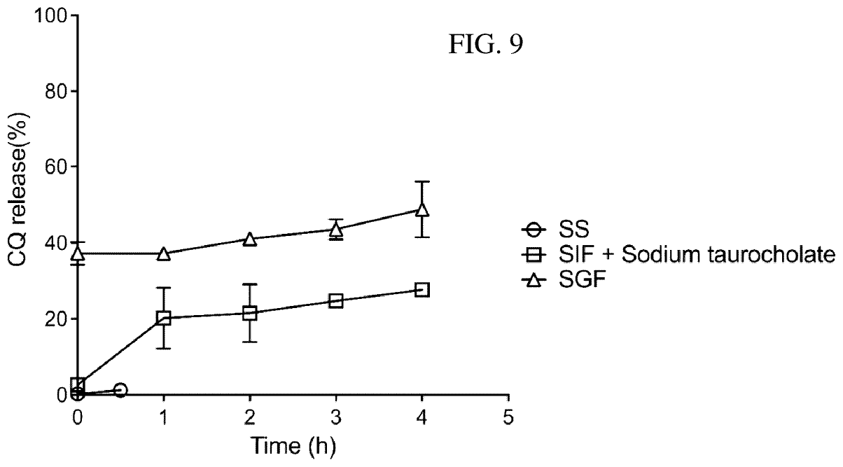

In an embodiment, the MLV-API drug formulation of the present invention retains up to about 90% to complete retention such as about 95%, about 96%, about 97%, about 98%, about 99% or complete retention of the API when residing within the physiological conditions of the oral cavity of a subject for up to 30 minutes as illustrated in Example 4 in connection with FIG. 9. In an embodiment, the MLV-CQ drug formulation retains up to about 90% to complete retention such as about up to about 95%, about 96%, about 97%, about 98%, about 99% or complete retention of the API within when residing within the physiological conditions of the oral cavity of a subject for up to 30 minutes as illustrated in Example 4 in connection with FIG. 9. The high encapsulation efficiency as well as the high API retention of the MLV of the present invention within a subject's oral cavity together effectively mask unpleasant taste of the API.

In an embodiment, as illustrated in Example 4 in connection with FIG. 9 the percentage of API released by the API loaded MLV of the present invention instantly or at instant burst in physiological conditions and temperature range equivalent to a subject's stomach is between about 30% to about 40% such as about 30%, about 32%, about 34% about 36%, about 38%, about 40% or any numbers or number ranges falling within these values. In an embodiment, instant burst is within about 1 to about 120 seconds such as about 1 second, about 2 seconds, about 5 seconds, about 15 seconds, about 30 seconds, about 45 seconds, about 60 seconds, about 80 seconds, about 100 seconds, about 120 seconds, or any numbers or number ranges falling within these values. In another embodiment, as illustrated in Example 4 in connection with FIG. 9 the percentage of the API released from the API loaded MLV of the present invention after about 4 hours at physiological conditions and temperature equivalent to the stomach of a subject is between about 40% to about 55% such as about 40%, about 42%, about 44%, about 46%, about 48%, about 50%, about 52% about 54% about 56%, about 58% about 60% or any numbers and number ranges falling within these values. In yet another embodiment, as illustrated in Example 4 in connection with FIG. 9, percentage of the API released from the API loaded MLV of the present invention after about 1 hour at physiological conditions and temperature equivalent to the intestine of a subject ranges between about 15% to about 30% such as about 15%, about 17%, about 20%, about 22%, about 25%, about 27%, about 30% or any numbers or number ranges falling within these values. And as illustrated in Example 4 in connection with FIG. 9, percentage of the API released from the API loaded MLV of the present invention after about 4 hours at physiological conditions and temperature equivalent to a subject's intestine is between about 15% to about 40% such as about 15%, about 17%, about 20%, about 22%, about 26%, about 28%, about 30%, about 32%, about 34%, about 36%, about 38%, about 40%, or any numbers or number ranges falling within these values.

In an embodiment, as illustrated in Example 4 in connection with FIG. 9 the percentage of CQ released by the CQ loaded MLV of the present invention instantly or at instant burst in physiological conditions and temperature equivalent to a subject's stomach is between about 30% to about 40% such as about 30%, about 32%, about 34% about 36%, about 38%, about 40% or any numbers or number ranges falling within these values. In an embodiment, instant burst is within about 1 to about 120 seconds such as about 1 second, about 2 seconds, about 5 seconds, about 15 seconds, about 30 seconds, about 45 seconds, about 60 seconds, about 80 seconds, about 100 seconds, about 120 seconds, or any numbers or number ranges falling within these values. In another embodiment, as illustrated in Example 4 in connection with FIG. 9 the percentage of the CQ released from the CQ loaded MLV of the present invention after about 4 hours at physiological conditions and temperature equivalent to the stomach of a subject is between about 40% to about 55% such as about 40%, about 42%, about 44%, about 46%, about 48%, about 50%, about 52% about 54% about 56%, about 58% about 60% or any numbers and number ranges falling within these values. In yet another embodiment, as illustrated in Example 4 in connection with FIG. 9, percentage of the CQ released from the CQ loaded MLV of the present invention after about 1 hour at physiological conditions and temperature equivalent to the intestine of a subject ranges between about 15% to about 30% such as about 15%, about 17%, about 20%, about 22%, about 25%, about 27%, about 30% or any numbers or number ranges falling within these values. And as illustrated in Example 4 in connection with FIG. 9 percentage of the CQ released from the CQ loaded MLV of the present invention after about 4 hours at physiological conditions and temperature equivalent to a subject's intestine is between about 15% to about 40% such as about 15%, about 17%, about 20%, about 22%, about 26%, about 28%, about 30%, about 32%, about 34%, about 36%, about 38%, about 40%, or any numbers or number ranges falling within these values.

In an embodiment, the MLV-API drug formulation of the present invention comprises API loaded MLV with diameter of about 50 to about 500 nm such as about 50, about 100, about 200, about 300, about 400, about 500 nm, or any numbers and number ranges falling within these values. In an embodiment, the MLV-CQ drug formulation of the present invention comprises MLV encapsulating CQ with diameter of about 200 to about 300 nm such as about 200, about 220, about 240, about 260, about 280, about 300 nm, or any numbers and number ranges falling within these values. In an embodiment, the MLV-NB drug formulation of the present invention comprises MLV encapsulating NB with diameter of about 150 to about 250 nm such as about 150, about 170, about 200, about 220, about 250, or any numbers and number ranges falling within these values. In an embodiment, the MLV-HCQ drug formulation of the present invention comprises MLV encapsulating HCQ with diameter of about 170 to about 270 nm such as about 170, about 190, about 210, about 230, about 250, about 270 nm, or any numbers and number ranges falling within these values.

In another embodiment, the MLV-API drug formulation of the present invention comprises API loaded MLV having polydispersity index (PDI) of about 0.3 to about 0.7 such as about 0.3, about 0.35, about 0.4, about 0.45, about 0.5, about 0.55, about 0.6, about 0.65, about 0.7 or any numbers and number ranges falling within these values. In another embodiment, the MLV-CQ drug formulation of the present invention comprises CQ loaded MLV having PDI of about 0.3 to about 0.4 such as about 0.3, about 0.32, about 0.34, about 0.36, about 0.38, about 0.4 or any numbers and number ranges falling within these values. In another embodiment, the MLV-NB drug formulation of the present invention comprises NB loaded MLV having PDI of about 0.4 to about 0.6 such as about 0.4, about 0.42, about 0.45, about 0.47, about 0.50, about 0.52, about 0.55, about 0.57, about 0.60 or any numbers and number ranges falling within these values. In another embodiment, the MLV-HCQ drug formulation of the present invention comprises HCQ loaded MLV having PDI of about 0.4 to about 0.6 such as about 0.4, about 0.42, about 0.45, about 0.47, about 0.50, about 0.52, about 0.55, about 0.57, about 0.60 or any numbers and number ranges falling within these values.

In yet another embodiment, the MLV-API drug formulation of the present invention comprises API loaded MLV with neutral or near neutral overall electrical charge. In an embodiment, the MLV drug formulation of the present invention has a zeta potential of about −15 to about 15 mV such as about −15, about −10, about −7.5, about −5, about −2.5, about 0, about 2.5, about 5, about 7.5, about 10, about −12.5, about 15 mV or any numbers and number ranges falling within these values. In an embodiment, the MLV-CQ drug formulation of the present invention comprises API loaded MLV with neutral or near neutral overall electrical charge at about −5 mV. In an embodiment, the MLV-NB drug formulation of the present invention comprises NB loaded MLV with neutral or near neutral overall electrical charge at about −4 mV. In an embodiment, the MLV-HCQ drug formulation of the present invention comprises HCQ loaded MLV with neutral or near neutral overall electrical charge at about −3 mV.

In an embodiment, the MLV-API drug formulation of the present invention comprises a lower pH within the interior of the MLV compared to the pH of the aqueous basic solution in which the MLV reside. In an embodiment, the pH within the MLV is about 1.0 to about 5.0 such as about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0 or any numbers or number ranges falling within these values. In an embodiment, the pH of the aqueous basic solution is about 7.2 to about 12.0 such as about 7.2, about 7.4, about 7.6, about 7.8, about 8.0, about 8.5, about 9.0, about 9.5, about 10.0, about 10.5, about 11.0, about 11.5, about 12.0, or any numbers and number ranges falling within these values. In an embodiment, the pH within the MLV is about 1.0 to about 3.0 such as about 1.0, about 1.2, about 1.4, about 1.6, about 1.8, about 2.0, about 2.2, about 2.4, about 2.6, about 2.8, about 3.0 or any numbers or number ranges falling within these values. In an embodiment, the pH of the aqueous basic solution is about 8.0 to about 10.0 such as about 8.0, about 8.5, about 9.0, about 9.5, about 10.0 or any numbers and number ranges falling within these values.

In an embodiment, the MLV-CQ drug formulation of the present invention comprises a lower pH within the interior of the MLV compared to the pH of the aqueous basic solution in which the MLV reside. In an embodiment, the pH within the MLV is about 1.0 to about 5.0 such as about 1.0, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0 or any numbers or number ranges falling within these values. In an embodiment, the pH of the aqueous basic solution is about 7.2 to about 12.0 such as about 7.2, about 7.4, about 7.6, about 7.8, about 8.0, about 8.5, about 9.0, about 9.5, about 10.0, about 10.5, about 11.0, about 11.5, about 12.0, or any numbers and number ranges falling within these values. In an embodiment, the pH within the MLV-CQ is about 1.0 to about 3.0 such as about 1.0, about 1.2, about 1.4, about 1.6, about 1.8, about 2.0, about 2.2, about 2.4, about 2.6, about 2.8, about 3.0 or any numbers or number ranges falling within these values. In an embodiment, the pH of the aqueous basic solution is about 8.0 to about 10.0 such as about 8.0, about 8.5, about 9.0, about 9.5, about 10.0 or any numbers and number ranges falling within these values.

In an embodiment, the MLV-NB drug formulation of the present invention comprises a lower pH within the interior of the MLV compared to the pH of the aqueous basic solution in which the MLV reside. In an embodiment, the pH within the MLV is about 1 to 5 such as about 1, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0 or any numbers or number ranges falling within these values. In an embodiment, the pH of the aqueous basic solution is about 7.2 to about 12.0 such as about 7.2, about 7.4, about 7.6, about 7.8, about 8.0, about 8.5, about 9.0, about 9.5, about 10.0, about 10.5, about 11.0, about 11.5, about 12.0, or any numbers and number ranges falling within these values. In an embodiment, the pH within the MLV-NB is about 1.0 to about 3.0 such as about 1.0, about 1.2, about 1.4, about 1.6, about 1.8, about 2.0, about 2.2, about 2.4, about 2.6, about 2.8, about 3.0 or any numbers or number ranges falling within these values. In an embodiment, the pH of the aqueous basic solution is about 8.0 to about 10.0 such as about 8.0, about 8.5, about 9.0, about 9.5, about 10.0 or any numbers and number ranges falling within these values.

In an embodiment, the MLV-HCQ drug formulation of the present invention comprises a lower pH within the interior of the MLV compared to the pH of the aqueous basic solution in which the MLV reside. In an embodiment, the pH within the MLV is about about 1 to 5 such as about 1, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0 or any numbers or number ranges falling within these values. In an embodiment, the pH of the aqueous basic solution is about 7.2 to about 12.0 such as about 7.2, about 7.4, about 7.6, about 7.8, about 8.0, about 8.5, about 9.0, about 9.5, about 10.0, about 10.5, about 11.0, about 11.5, about 12.0, or any numbers and number ranges falling within these values. In an embodiment, the pH within the MLV-HCQ is about 1.0 to about 3.0 such as about 1.0, about 1.2, about 1.4, about 1.6, about 1.8, about 2.0, about 2.2, about 2.4, about 2.6, about 2.8, about 3.0 or any numbers or number ranges falling within these values. In an embodiment, the pH of the aqueous basic solution is about 8.0 to about 10.0 such as about 8.0, about 8.5, about 9.0, about 9.5, about 10.0 or any numbers and number ranges falling within these values.

In an embodiment, the MLV-API drug formulation of the present invention further comprises cryoprotectants comprising sucrose, dextrose, glucose, trehalose, ethylene glycols, propylene glycols, dextran, cyclodextrane, arginine, P188, polyvinylpyrrolidone, glycerol, sorbitol, albumin, mannitol or a combination thereof. In an embodiment, the MLV drug formulation of the present invention comprises cryoprotectants at a concentration of about 15, 20, 25, 30, 35, 40, 45%, or any numbers and number ranges falling within these values.

In an embodiment, the MLV-API drug formulation of the present invention may further comprise two or more API wherein the first API is encapsulated within the MLV of the present invention and the second compound is within the aqueous basic solution outside of the MLV. In an embodiment, the second compound comprises an API different from the first API so that the oral drug formulation of the present invention can provide combination therapy of two or more drugs. In an embodiment, weak bases such as primaquine, mefloquine, paracetamol can be loaded into MLVs whereas weak acids can stay in the exterior phase. In another embodiment, the MLV drug formulation of the present invention further comprises a third compound which comprises an adjuvant residing within the MLV or in the aqueous basic solution outside of the MLV.

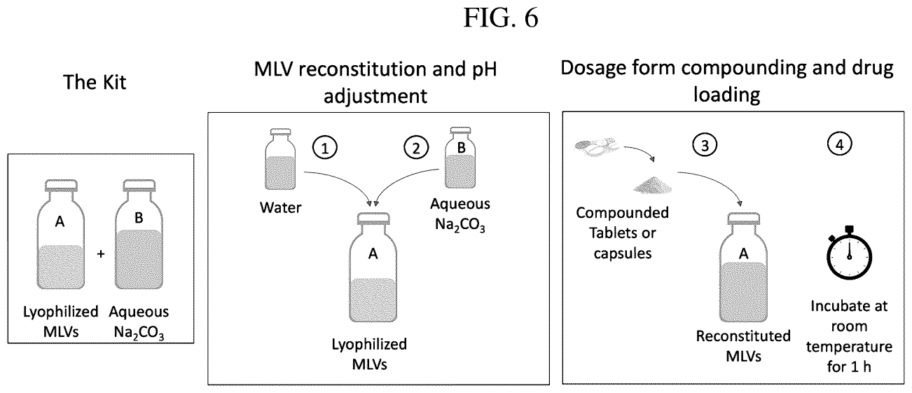

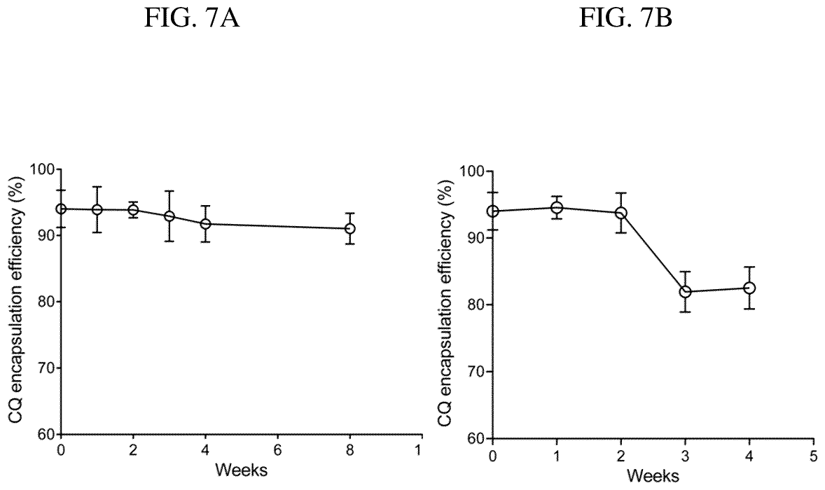

The present invention also provides a MLV-API drug formulation kit comprising 1. an API in powder form or any form capable of being made into powder form such as a tablet capable of being ground into powder form, 2. an aqueous basic solution and 3. lyophilized MLVs not encapsulating any API wherein the three components are kept and stored separately. In an embodiment, the lyophilized MLV can be stored at room temperature for at least 4, 5, 6, 7, or 8 weeks and realize encapsulation efficiency of about 90% or more when made into MLV-API drug formulation from the kit of the present invention as illustrated in Example 2 in connection with FIG. 7A. In an embodiment, the MLV-API drug formulation of the present invention can be prepared by mixing the three components as illustrated in FIG. 6 without dialysis or extrusion procedures. In an embodiment, the MLV-API drug formulation of the present invention can be prepared by mixing by hand the three components when API is made into powder form as illustrated in FIG. 6 without dialysis or extrusion procedures wherein mixing by hand comprises combining all three components in a single container and lightly shaking the container by hand for about 1 to about 10 minutes such as about 1 minute, about 2 minutes, about 5 minutes, about 7 minutes, about 10 minutes or any time period or time ranges falling within these values. In an embodiment, the mixing by hand comprises combining all three components in a single container only without shaking the container. In an embodiment, the mixing by hand comprises combining all three components in a single container and vigorously shaking the container by hand for about 1 to about 10 minutes such as about 1 minute, about 2 minutes, about 5 minutes, about 7 minutes, about 10 minutes or any time period or time ranges falling within these values. In an embodiment, the mixing by hand comprises combining all three components in a single container and shaking the container by hand until the solution becomes homogenous.

In an embodiment, bases that may be used instead or in combination with sodium carbonate may include but are not limited to potassium carbonate, calcium carbonate, magnesium carbonate, sodium bicarbonate, potassium bicarbonate, calcium bicarbonate, magnesium bicarbonate, sodium hydroxide, potassium hydroxide, calcium hydroxide, magnesium hydroxide and ammonia. In an embodiment, the API comprises chloroquine (CQ), CQ derivative (e.g., hydrochloroquine (HCQ)), clonidine, clonidine derivative, imatinib, propranolol, pilocarpine, meloxicam, aripiprazole, chlorpromazine, R848, imiquimod, mefloquine and primaquine or a pharmaceutically acceptable salt thereof.

In an embodiment, the encapsulation efficiency of the MLV-API drug formulation prepared using the kit of the present invention can be maintained at >80% after about 20 to about 34 days such as about 20 days, about 22 days, about 24 days, about 26 days, about 28 days, about 30 days, about 32 days, about 34 days or any number of number ranges falling within these values storing at about 4° C. In an embodiment, the encapsulation efficiency of the MLV-API drug formulation of the present invention prepared using the kit of the present invention can be maintained at >94% after about 12 to about 24 days such as about 12 days, about 14 days, about 16 days, about 18 days, about 20 days, about 22 days, about 24 days or any number of number ranges falling within these values storing at about 4° C.

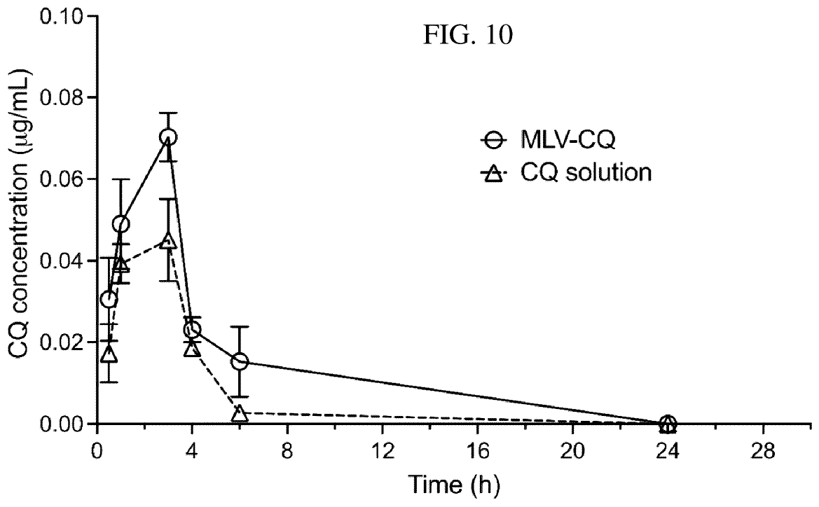

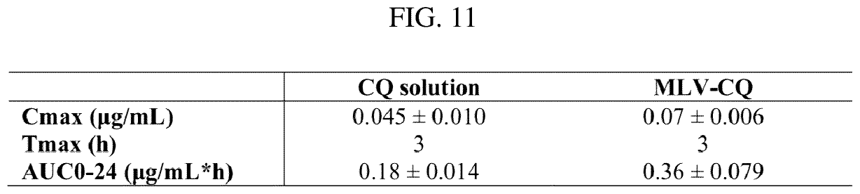

In an embodiment, the MLV-API drug formulation of the present invention is capable of enhancing the absorption of the encapsulated drug. In an embodiment, the 24 hour AUC of the formulation of the present invention is more than twice as high as free form API while Tmax remains the same as shown in FIGS. 10 and 11. In an embodiment, the Cmax of the MLV of the present invention is about 1.2, 1.4, 1.6, 1.8, 2.0, 2.2, 2.4, 2.6, 2.8, 3.0-fold higher than the drug absorption of the free-form drug in a subject while Tmax remains the same as shown in FIGS. 10 and 11.

In an embodiment, the molar ratio of cholesterol to lipid of the any embodiment of the MLV of the present invention may be about 1:0.2, 1:0.4, 1:0.6, 1:0.8, 1:1, 1:1.2, 1:1.4, 1:1.6, 1:1.8, 1:2, 1:2.2 or any ratio in between. In an embodiment, the lipid comprises DSPC, DOPC, DOPS, DPPS, DSPS, DOPE or a combination thereof.

In an embodiment, the any embodiment of the MLV-API drug formulation of the present invention is made without extrusion and/or dialysis procedures.

In an embodiment, any embodiment of the MLV-API drug formulation of the present invention may comprise but is not limited to one or more of the following components: phospholipids, lysolipids, lysophospholipids, sphingolipids, pegylated lipids, phosphatidylserines, phosphatidylglycerols, phosphatidylinositols (not limited to a specific sugar), fatty acids, sterols, caprylic acid, capric acid, triglyceride, medium-chain triglyceride (MCT), lauric acid, myristic acid, palmitic acid, steric acid, corn oil, peanut oil, oleic acid, Span 80, any other edible oil, Tween, Gelucire 44/14, Gelucire 50/13, Brij 35, Brij 58, polyoxy 20 monostearate, polyoxy 40 monostearate, lecithin, tocophersolan (TPGS), labrasol ALF, ethanol, glycerol, PEG 300-4000, PEG 8000, Transcutol P, Soluphor P, in any ratio deemed acceptable by those skilled in the art.

The present invention further comprises a method of preparation of the MLV-API drug formulation of the present invention comprising the steps of:
i. Preparing MLV and establishing pH gradient:
   a. Forming MLV by hydrating organic components with an acidic solution. In an embodiment, the acidic solution may comprise citric acid, tartaric acid, malic acid, acetic acid, oxalic acid, ascorbic acid, sodium acetate or a combination thereof. In an embodiment, the organic components of the MLV comprises DSPC, DOPC, DOPS, DPPS, DSPS, DOPE, Chol, phospholipids, lysolipids, lysophospholipids, sphingolipids, pegylated lipids, phosphatidylserines, phosphatidylglycerols, phosphatidylinositols (not limited to a specific sugar), or a combination thereof.
   d. Adding the MLV created in step i a to an aqueous basic solution to form an aqueous basic solution containing the MLV to establish a pH gradient between the interior of the MLV and the aqueous basic solution to facilitate active loading of API in step ii. In an embodiment, the aqueous basic solution may comprise sodium carbonate, potassium carbonate, calcium carbonate, magnesium carbonate, sodium bicarbonate, potassium bicarbonate, calcium bicarbonate, magnesium bicarbonate, sodium hydroxide, potassium hydroxide, calcium hydroxide, magnesium hydroxide, ammonia or a combination thereof.
ii. Active load API:
   a. Dissolving an API in powder form in water to form an API solution. In an embodiment, the water comprises distilled water. In an embodiment, the step of grinding the API into powder form may be performed first.
   b. Mixing the API solution of step ii a with the MLV in aqueous basic solution prepared in step i to form an active loading solution.
   c. Incubate the active loading solution of step ii b to effect active loading of the API into the MLV. In an embodiment, the incubation temperature may range from about 15° C. to about 70° C. such as about 15° C., about 20° C., about 25° C., about 30° C., about 35° C., about 40° C., about 45° C., about 50° C., about 55° C., about 60° C., about 65° C., about 70° C. or any temperatures or temperature ranges falling within these values, and the incubation time may range from about 5 min to about 2 h such as about 5 min, about 10 min, about 20 min, about 30 min, about 40 min, about 50 min, about 60 min, about 70 min, about 80 min, about 90 min, about 100 min, about 110 min, about 120 min or any period of time or time ranges falling within these values. In an embodiment, the incubation temperature may range from about 50° C. to about 70° C. such as about 50° C., about 55° C., about 60° C., about 65° C., about 70° C. or any temperatures or temperature ranges falling within these values and the incubation time may range from about 40 min to about 80 min such about 40 min, about 50 min, about 60 min, about 70 min, about 80 min any period of time or time ranges falling within these values.

In an embodiment, the method of preparing MLV-API of the present invention further comprises a step i b of adding cryoprotectants to the solution containing the MLV of step i a and freeze drying the MLV formed in step i a. In an embodiment, the freeze drying conditions are at about −80° C. to about −100° C. such as about −80° C., about −85° C., about −90° C., about −95° C., about −100° C. or any temperatures or temperature ranges falling within these values at about 0.05 mBar to about 0.2 mBar overnight such as about 0.05 mBar, about 0.1 mBar, about 0.15 mBar, about 0.2 mBar or any pressures or pressure ranges falling within these values. In an embodiment, the cryoprotectants of step i b comprises sucrose, dextrose, trehalose or a combination thereof. In an embodiment, the concentration of cryoprotectants within the solution of step i b is about 15% to 45% such as about 15, about 20, about 25, about 30, about 35, about 40, about 45% or any concentrations or concentration ranges falling within these values. In an embodiment, the method of preparing MLV-API drug formulation of step i of the present invention further comprises the step i c of storing the lyophilized MLV of step i b at 4° C. for up to 8 weeks prior to performing the rest of the steps i d and steps ii a-c and still attaining encapsulating efficiency of over about 80%, about 90% or about 95% at D/L ratio of between about 5% to about 20% by weight percentage in the active loading solution such as about 5%, about 10%, about 15%, about 20% or any percentages or percentage ranges falling within these values in the active loading solution after the storage when the rest of the preparation steps are carried out.

Any embodiment of the method for preparing the MLV-API drug formulation of the present invention does not comprise membrane extrusion step and dialysis step. The liposomal technology that does not require membrane extrusion step and dialysis would be substantially easier and economical for preparation and scale-up manufacturing.

In an embodiment, the hydrating step in step i a wherein the organic components are in the form of a thin film before the hydration. In an embodiment, the organic components in step i a comprises cholesterol and a lipid at a molar ratio of about 1:0.2 to about 1:2.2 such as 1:0.2, 1:0.4, 1:0.6, 1:0.8, 1:1, 1:1.2, 1:1.4, 1:1.6, 1:1.8, 1:2, 1:2.2 or any numbers and number ranges falling within these values. In an embodiment, the hydration step in step i a of the present invention comprises adding an acid solution of pH about 1.0 to about 5.0 such as about 1, about 1.2, about 1.4, about 1.6, about 1.8, about 2.0, about 2.2, about 2.4, about 2.6, about 2.8, about 3.0, about 3.2, about 3.4, about 3.6, about 3.8, about 4.0, about 4.2, about 4.4, about 4.6, about 4.8, about 5.0 or any numbers or number ranges falling within these values at a concentration of about 50 to about 500 mM such as about 50, about 100, about 150, about 200, about 250, about 300, about 350, about 400, about 450, about 500, about 550 mM or any concentration and any concentration range falling within these values. In an embodiment, the MLV preparation step in step i d of the present invention comprises adding a basic solution of pH 7.2 to about 12.0 such as about 7.2, about 7.4, about 7.6, about 7.8, about 8.0, about 8.5, about 9.0, about 9.5, about 10.0, about 10.5, about 11.0, about 11.5, about 12.0 or any pH or pH ranges falling within these values at a concentration of about 50 to about 550 mg/mL such as about 50 mg/mL, about 100 mg/mL, about 150 mg/mL, about 200 mg/mL, about 250 mg/mL, about 300 mg/mL, about 350 mg/mL, about 400 mg/mL, about 450 mg/mL, about 500 mg/mL, about 550 mg/mL or any concentration and any concentration range falling within these values. In an embodiment, the hydration step in step i a of the present invention comprises adding an acid solution of pH about 1.0 to about 3.0 such as about such as about 1, about 1.2, about 1.4, about 1.6, about 1.8, about 2.0, about 2.2, about 2.4, about 2.6, about 2.8, about 3.0 or any numbers or number ranges falling within these values at a concentration of about 50 to about 500 mM such as about 50, about 100, about 150, about 200, about 250, about 300, about 350, about 400, about 450, about 500, about 550 mM or any concentration and any concentration range falling within these values. In an embodiment, the MLV preparation step in step i b of the present invention comprises adding a basic solution of pH 7.2 to about 10.0 such as about 7.2, about 7.4, about 7.6, about 7.8, about 8.0, about 8.5, about 9.0, about 9.5, about 10.0 or any pH or pH ranges falling within these values at a concentration of about 50 to about 550 mg/mL such as about 50 mg/mL, about 100 mg/mL, about 150 mg/mL, about 200 mg/mL, about 250 mg/mL, about 300 mg/mL, about 350 mg/mL, about 400 mg/mL, about 450 mg/mL, about 500 mg/mL, about 550 mg/mL or any concentration and any concentration range falling within these values.

In another embodiment, MLV-API formulation of the present invention is prepared by combining the alternative components of caprylic acid, capric acid, triglyceride, medium-chain triglyceride (MCT), lauric acid, myristic acid, palmitic acid, steric acid, corn oil, peanut oil, oleic acid, Span 80, any other edible oil, Tween, Gelucire 44/14, Gelucire 50/13, Brij 35, Brij 58, polyoxy 20 monostearate, polyoxy 40 monostearate, lecithin, tocophersolan (TPGS), labrasol ALF, ethanol, glycerol, PEG 300-4000, PEG 8000, Transcutol P, Soluphor P, or a combination thereof.

In an embodiment, the active loading step in step ii of the present invention comprises an API wherein the API comprises weak base drugs. In an embodiment, the API of the active loading step in step ii of the present invention comprises CQ, CQ derivative, clonidine, clonidine derivative, imatinib, propranolol, pilocarpine, meloxicam, aripiprazole, chlorpromazine, R848, imiquimod, mefloquine and primaquine or a pharmaceutically acceptable salt thereof.

In an embodiment, the MLV drug formulation prepared using the method of the present invention may have a D/L ratio of up to about 5 wt % to about 30 wt % such as about 5 wt %, about 10 wt %, about 15 wt %, about 20 wt %, about 25 wt %, about 30 wt % or any wt % or wt % ranges falling within these values.

The present invention also provides a method of treatment of illness using any embodiments of the MLV drug formulation of the present invention. In an embodiment, the method of treatment using any embodiments of the MLV drug formulation of the present invention comprises treating a subject that is sensitive to the unpleasant taste of the drug in its free form. In another embodiment, the method of treatment using any embodiments of the MLV drug formulation of the present invention comprises treating a subject that has difficulty in swallowing. In an embodiment, the method of treatment using any embodiments of the MLV drug formulation of the present invention comprises treating a child. In an embodiment, the illness to be treated using any embodiment of the MLV drug formulation of the present invention comprises rheumatoid arthritis, lupus erythematosus and malaria. In another embodiment, the illness to be treated using any embodiment of the MLV drug formulation of the present invention comprises high blood pressure, ADHD, drug withdrawal, menopausal flushing, diarrhea, spasticity, and certain pain conditions.

The following examples should be illustrative only but are not meant to be limiting to the scope of the invention. Other generic and specific configurations will be apparent to those skilled in the art.

### Examples of CQ-, NB-, HCQ-Loaded MLV

### Materials and Methods

### Materials

1,2-distearoyl-sn-glycero-3-phosphatidylcholine (DSPC) was purchased from Avanti Polar lipids (Alabaster, AL). Cholesterol (Chol), chloroquine diphosphate (CQ), hydroxychloroquine sulfate (HCQ), Nile Blue (NB), citric acid monohydrate, sodium carbonate (Na₂CO₃), phosphoric acid, phosphate buffered saline (PBS), concentrated hydrochloric acid and sodium taurocholate hydrate were purchased from Sigma Aldrich (St. Louis, MO). Hydroxychloroquine tablets (200 mg/tablet) were purchased from Regency Medical Centre Pharmacy (Vancouver, BC, Canada). All chemical reagents and organic solvents were of analytical grade.

### Simulated Fluids

Simulated saliva (SS, pH 6.5) was prepared by titrating 10 mM PBS (pH 7.4) with 1 M phosphoric acid. Simulated gastric fluid (SGF, pH 1.2) and simulated intestinal fluid (SIF, pH 6.8) were both purchased from RICCA Chemical Company (Arlington, TX). Sodium taurocholate hydrate (3 mM) was added to the simulated intestinal fluid to mimic bile salt in the intestine.

### Preparation of Liposomal Multilamellar Vesicles (MLVs)

MLVs were prepared using the thin film hydration method as previously described.¹⁴ The mixture of cholesterol/DSPC (45/55, molar ratio) was dissolved at a total lipid concentration of 30 mg/mL in chloroform in a round-bottomed flask. Chloroform was removed using rotary evaporation at 60° C. and a rotation speed of 400 rpm to obtain a thin lipid film. The lipid film was dried under high vacuum for 30 min to remove residuals of chloroform. The film was then hydrated with 2 mL of 300 mM citric acid (pH 2). The MLV suspension was heated at 40° C. and sonicated for 30 min to obtain a homogeneous and milky liquid preparation. Particle size, polydispersity index (PDI) and zeta potential of the MLVs were measured by dynamic light scattering (DLS) using Zetasizer NanoZS (Malvern Instruments, Malvern, UK). For lyophilization, MLVs prepared in citric acid (pH 2) with 30% sucrose were aliquoted into 200 μL and freeze dried overnight at −90° C. and 0.12 mBar, using FreeZone® Triad™ freeze dry system (LABCONCO, Kansas City, MO). The samples were sealed and stored at room temperature for stability monitoring.

### Active Loading of Drugs

Approximately 34-40 μL of sodium carbonate (300 mg/mL) was added to 200 μL of the liposomal MLVs to bring up the exterior pH to 8-10. CQ and HCQ-powder were dissolved in distilled water at a final concentration of 10 mg/mL. HCQ-tablet (200 mg) was first ground into fine powder and dissolved in distilled water at 10 mg/mL. Loading of the drugs was performed at drug-to-lipid (D/L) ratios ranging from 5%-30% (w/w) by mixing the drug solution with the MLVs containing a pH gradient, followed by incubation at room temperature for 1 h and quenching on ice for 2 min to terminate the loading process. Un-encapsulated drug was removed by size exclusion chromatography (SEC) on a Sephadex-G25 column (GE Healthcare, Milwaukee, WI). Drug and lipid concentrations in samples were quantitatively analyzed by UPLC as described below. The drug encapsulation efficiency (EE) was calculated by the following equation where D/L_{before} and D/L_{after} denote drug-to-lipid ratios before and after SEC, respectively.

$$Encapsulation efficiency ( \% ) = \frac{{( D / L )}_{After}}{{( D / L )}_{Before}} \times 100 \qquad ( 1 )$$

Alternatively, the MLVs were centrifuged at 10,000 g for 10 min and the supernatant containing unencapsulated drug was analyzed by UPLC. The result was then compared with the total drug concentration in the MLVs to obtain the EE.

### Cryogenic Transmission Electron Microscopy (CryoTEM)

Empty and CQ-loaded MLVs (30 mg lipid/mL) at 20% D/L ratio (w/w) were deposited onto a glow-discharged copper grid, vitrified using a FEI Mark IV Vitrobot (FEI, Hillsboro, OR, USA), and imaged using a 200 kV Glacios microscope equipped with a Falcon III camera at the UBC High Resolution Macromolecular Cryo-Electron Microscopy facility (Vancouver, BC, Canada).

### Ultraperformance Liquid Chromatography (UPLC)

Concentrations of drugs and lipids were simultaneously quantified by a ACQUITY UPLC H-Class System (Waters, Milford, MA)¹⁵. The mobile phase was composed of solvent A: 0.1% trifluoroacetic acid (TFA) in water and solvent B: 0.1% TFA in methanol. CQ and HCQ were quantified by a photodiode array (PDA) detector at wavelengths of 342 and 342, respectively. DSPC and Chol were quantified by an evaporative light scattering (ELS) detector. Concentrations of drugs and lipids were measured by integrating the peak area under the curve and compared to the standard curves. For plasma CQ quantitation, a Waters QDa mass spectrometry detector was used. Ionization was carried out at 250° C. (source temperature) using a cone voltage of 65 V, and concentration of CQ was determined by integrating the single ion recognition (SIR) peak for the singly charged molecular ion (m/z 319.8) acquired at a capillary voltage of 0.5 V.

### Storage Stability Study

MLVs prepared in citric acid with 30% sucrose were lyophilized and stored at room temperature. At selected timepoints (1, 2, 3, 4 and 8 weeks), the lyophilized MLVs was reconstituted with Milli-Q water. Sodium carbonate was then added into the MLVs to create a transmembrane pH gradient as described above, followed by loading with CQ at a D/L of 20 wt %. The drug loading efficiency was measured using the centrifugation method. The prepared CQ-loaded MLV drug formulation was stored in the liquid form at 4° C. At different time points, CQ encapsulation efficiency was determined using the centrifugation method.

### In Vitro Drug Release

MLV-CQ drug formulation of the present invention with MLVs loaded with CQ at 20% D/L (w/w) were mixed with different simulated gastrointestinal fluids at 1:1 (v/v), incubated at 37° C. with shaking (150 rpm). At selected time points, 200 μL of the mixture was collected, and the released drug was removed by SEC. Drug and lipid concentrations of MLVs, before and after SEC, were measured using UPLC. Drug release (%) was obtained by equation (2):

$$Drug release ( \% ) = ( 1 - \frac{{( D / L )}_{After}}{{( D / L )}_{Before}} ) \times 100 \qquad ( 2 )$$

### Animals

Female CD1 mice (18-20 g, 6-7 weeks old) were purchased from The Jackson Laboratory (Bar Harbor, ME). All the in vivo studies were conducted in accordance with an established protocol (A18-0177) approved by the Animal Care Committee of the University of British Columbia (Vancouver, BC, Canada).

### Pharmacokinetics

MLV-CQ drug formulation of the present invention or free CQ (dissolved in saline) was administered through oral gavage to mice at 10 mg CQ/kg (50 mg lipid/kg). At 0.5, 1, 3, 4, 6 and 24 h, blood was collected through either the saphenous vein or cardiac puncture and was quickly transferred into an EDTA-coated tube. Plasma was isolated by centrifugation at 4° C. for 10 min at 10,000 g. Forty-five L of plasma was mixed with 300 μL ethanol, vortexed for 30 s, placed on ice for 30 min, and centrifuged twice at 12,500 rpm for 5 min. The supernatant (280 μL) was collected, lyophilized, and reconstituted in 45 μL ethanol. Ten L of the sample was then injected into the UPLC to measure CQ concentration. Pharmacokinetics parameters and AUC were analyzed using PK solver¹⁶ and GraphPad Prisim version 8.0 (GraphPad Software, San Diego, CA, USA), respectively.

### Statistical Analysis

All data are expressed as mean±SD. Statistical analysis was performed with GraphPad Prism version 8.0. Comparison between two groups were made by unpaired t-test, and one-way ANOVA was used for comparisons among three or more groups.

### Example 1. Preparation of Drug-Loaded MLVs

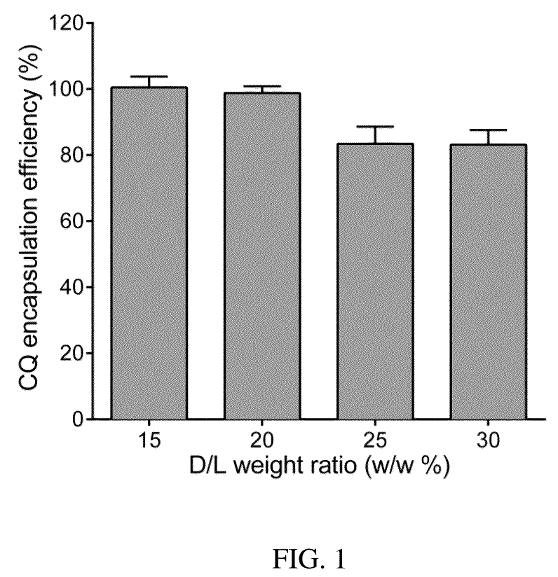

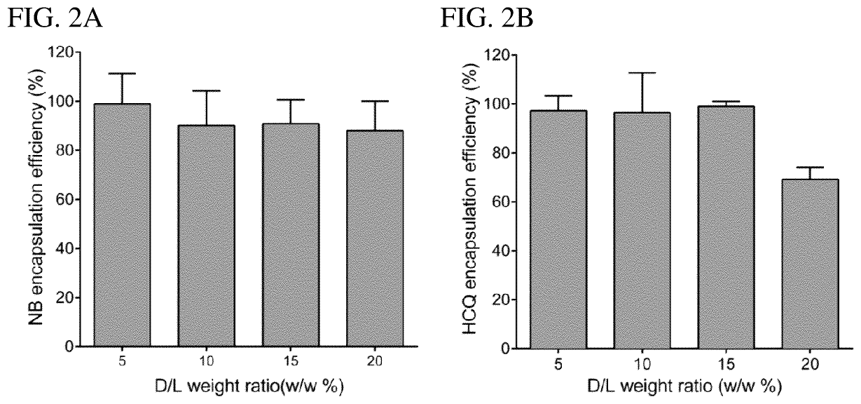

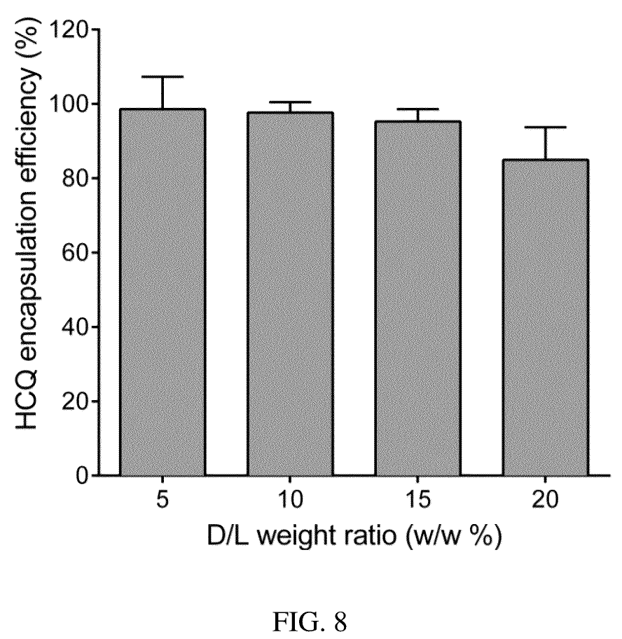

MLV liposomes were spontaneously formed by hydrating DSPC and cholesterol with 300 mM citric acid (pH 2), without the tedious membrane extrusion process or dialysis process. A transmembrane pH gradient was created by adding aqueous Na₂CO₃ to the MLVs to bring the external pH to 8-10. To demonstrate whether this technology could provide active and complete loading of CQ, the gradient MLVs were mixed with CQ at a range of D/L, incubated at room temperature for 1 h before analysis of EE. As shown in FIG. 1, complete (>95%) drug encapsulation was determined at D/L of 15-20% (w/w). Drug entrapment efficiency decreased to ~80% as the D/L increased to 25% (w/w) (FIG. 1), indicating saturation of drug loading. It was long thought that drugs could not be actively loaded into MLVs due to the limited aqueous space between the lipid bilayers. In the preparation method of the present invention, we disclose a method for active loading of API into MLV via pH gradient. Additionally, the MLV drug formulation of the present invention was prepared using simple and scalable techniques and provided complete encapsulation for weak-base drugs. Besides CQ, we successfully encapsulated other weak-base small molecules such as HCQ, NB and clonidine into the MLV of the present invention at the same D/L (FIGS. 2 and 8) demonstrating that this could serve as a platform technology.

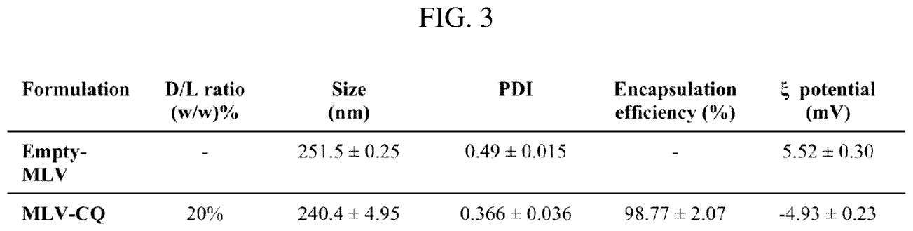

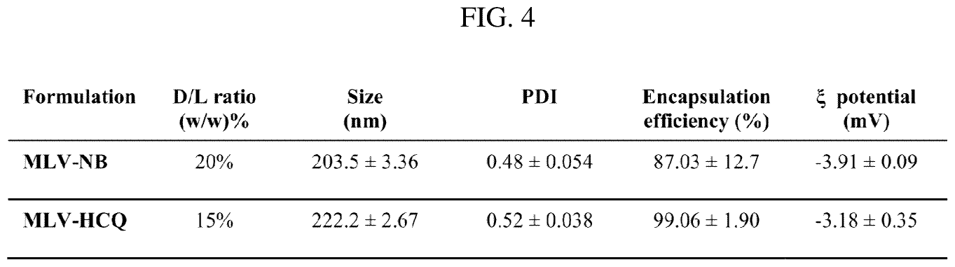

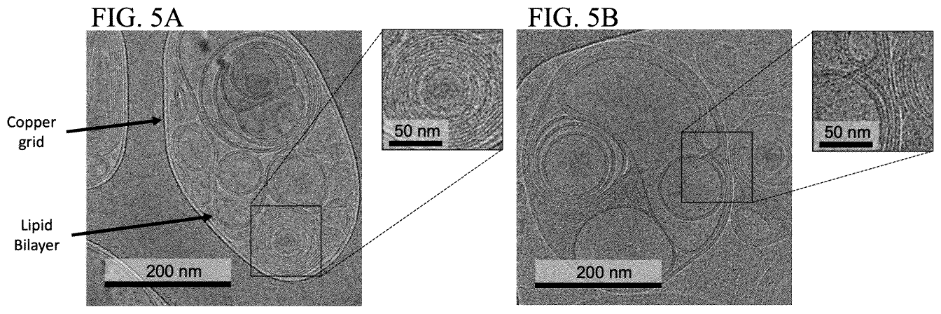

Empty and CQ-loaded MLVs were characterized by DLS and displayed comparable size (~250 nm), PDI (0.3-0.5) and zeta potential (±5 mV) (FIG. 3). The D/L ratio, size, PDI, encapsulation efficiency, and zeta potential of MLV-NB and MLV-HCQ were also characterized (FIG. 4). The high PDI was expected as there was no membrane extrusion in the preparation to control the particle size. The relatively small size of these MLV could be caused by the gradient introducing method that might promote lipid rearrangements. The multilamellar morphology of MLVs and variable particle size distribution were further confirmed by cryo-TEM images (FIG. 5). Unlike the previously reported SUV-Mef structure⁶, no electron-dense drug aggregates were shown inside the MLVs.

### Example 2. Development of a User Kit for Simple Preparation of Stable Liquid Drug Formulation

We envisioned that this MLV platform could be used as a universal taste-masking formulation for weak-base drugs. The drug-free MLVs in the lyophilized form will be provided along with aqueous Na₂CO₃ in the pharmacy or to the care giver as a kit complete with user instructions. First, the MLVs will be reconstituted with a fixed amount of water, followed by an addition of a fixed volume of the aqueous Na₂CO₃ to increase the external pH to 8-10. A fixed amount of the drug powder (15-20 wt %) will be mixed with the MLVs and then incubated at room temperature for about 1 h before use, as shown in FIG. 6.

To examine the feasibility of this approach, we lyophilized MLVs prepared in 300 mM citric acid (pH 2) with 30% sucrose into dry powder, which was then stored at room temperature for up to 8 weeks. At different time points, the lyophilized MLVs were reconstituted in MilliQ water, adjusted for pH (8-10) using aqueous Na₂CO₃, mixed with 20 wt % CQ, incubated at room temperature for 1 h, and measured for drug encapsulation efficiency using the centrifugation method. As shown in FIG. 7A, the lyophilized MLVs could be stored at room temperature for at least 2 months and still provide >90% encapsulation efficiency for CQ, indicating that the lyophilized MLV product exhibited good storage stability at room temperature and was ready for use to prepare a child-friendly liquid formulation by following simple procedures. Additionally, the prepared MLV-CQ was stable for 2 weeks upon storage at 4° C. As shown in FIG. 7B, the CQ encapsulation efficiency was maintained at >94% for 2 weeks at 4° C., but decreased to ~80% in 3 weeks. The data suggest that once a pediatric formulation is prepared using this MLV kit, patients can store this formulation at 4° C. for up to 2 weeks.

### Example 3. Compounding a Hydroxychloroquine Tablet into a Liquid Formulation Using the MLV Drug Formulation Kit

Next, we examined whether the MLV drug formulation kit could be used to compound a commercially available HCQ tablet into a liquid formulation. CQ tablets are not available in Canada, and therefore, we acquired HCQ tablets as an alternative for the study. Extemporaneous compounding of an adult dosage form into a liquid preparation is commonly practiced by pharmacists and care givers when a child-friendly dosage form is not commercially available. As discussed earlier, this common practice often leads to poor medication adherence due to inefficient taste masking.

The tablet (200 mg HCQ/tablet) was ground into fine powder and dissolved in MilliQ water at a final concentration of 10 mg HCQ/mL. MLVs containing a pH gradient were prepared and mixed with the HCQ suspension at a range of D/L ratios (5-20 wt %), followed by incubation at room temperature for 1 h before measurement of HCQ encapsulation efficiency. As shown in FIG. 8, MLV-HCQ prepared at a D/L of 15 wt % displayed almost complete drug encapsulation efficiency, while the efficiency decreased to ~85% when the D/L increased to 20 wt %. The data suggest that the MLV drug formulation kit could be employed to support effective compounding of a child-friendly liquid formulation from an adult tablet following easy procedures.

### Example 4. In Vitro Drug Release Studies

We then examined whether CQ could be released from the MLV drug formulation of the present invention in gastrointestinal fluids after oral delivery and performed drug release studies in simulated saliva (SS), simulated gastric fluid (SGF), and simulated intestinal fluid (SIF) supplemented with 3 mM sodium taurocholate to mimic oral, gastric, and intestinal conditions, respectively. MLV-CQ were incubated with various simulated fluids at 37° C. while shaking at 150 rpm. Drug release was measured by comparing CQ concentration before and after SEC at the selected timepoints. As shown in FIG. 9, when incubating MLV-CQ drug formulation of the present invention with SS, there was no drug release for 30 min, indicating that CQ would be retained inside the MLVs within the oral cavity. As we have previously established, the unpleasant taste of a compound depends on the concentration of the free or released form of drug. As such, the data suggest that this MLV delivery system providing 100% retention of CQ would completely mask the bitter taste. Since the formulation transition time through the oral cavity would be short, we only monitored drug release for 30 min. We then examined drug release in SGF (pH 1.2). As shown in FIG. 9, the formulation displayed an instant burst release (~38%) upon incubation with SGF with a cumulative CQ release of ~50% after 4 h. This burst release can be explained by the extremely acidic pH in SGF, where the phosphate groups (pKa ~3) of the DSPC would be protonated, resulting in imbalance between the hydrophobicity and hydrophilicity of the phospholipid and ultimately causing instability in the lipid bilayer.⁶ Finally, CQ release was evaluated in SIF supplemented with 3 mM sodium taurocholate, a bile salt commonly found in the intestines. MLV-CQ displayed 20% drug release after 1 h incubation in SIF, and then the release gradually increased to 30% in 4 h, as shown in FIG. 9. The bile salt could act as a surfactant to disrupt the liposomal membrane for drug release as demonstrated and suggested previously.⁶ The data indicate that when MLV-CQ drug formulation of the present invention was administered orally, the MLV could retain CQ, preventing its contact with the taste buds and masking the bitter taste. After transition to the stomach and intestine, CQ could be effectively released in the gastrointestinal tract due to the acidic environment and the presence of bile salt surfactants.

### Example 5. Pharmacokinetics

A pharmacokinetics study was conducted to examine and compare the oral delivery of MLV-CQ and CQ aqueous solution in vivo. CD-1 mice were orally dosed with MLV-CQ (liquid) or CQ solution at 10 mg/kg, and the plasma was collected at 0.5, 1, 3, 4, 6 and 24 h post administration to measure CQ concentration. After oral administration, CQ concentration in plasma increased gradually, reaching Cₘₐₓ at 3 h and then was completely eliminated from plasma at 24 h, for both MLV-CQ and CQ solution. As shown in FIG. 10 and FIG. 11, the plasma profiles of MLV-CQ and CQ solution were comparable; however, MLV-CQ displayed 1.75 and 2-fold higher Cₘₐₓ and AUC, respectively, compared to CQ solution, suggesting enhanced drug absorption with MLVs. This data is in line with previous reports demonstrating that lipids are effective absorption enhancers.¹⁷

### Conclusion

We have developed a MLV platform technology for making child-friendly oral liquid formulations for weak-base drugs. This platform could be developed into a kit available to pharmacists and care-givers to support compounding of adult solid dosage forms into a pediatric formulation. There is a high demand for such solutions to address the issue of medication adherence that leads to failure of therapy in this special population.

### Examples of Clonidine-Loaded MLV

### Materials and Methods

### Materials

DSPC (18:0 PC) and cholesterol (≥99%) were purchased from Sigma-Aldrich (Oakville, ON, Canada). Clonidine hydrochloride (≥98%) and other general laboratory chemicals were purchased from VWR Scientific (Mississauga, ON, Canada).

### Hydration Buffer Solutions

Buffer A comprises 0.3 M citric acid solution (pH 1.6). Buffer B comprises 0.3 M sodium citrate buffer (pH 3.6). Buffer C comprises 0.3 M sodium citrate buffer (pH 5.5). Buffer D comprises 0.3 M ammonium sulfate buffer (pH 5.0).

### Preparation and Characterization of Clonidine-Loaded DSPC/Cholesterol Multilamellar Vesicles

Thin film hydration method was used for preparing clonidine-loaded DSPC/cholesterol multilamellar vesicles (MLVs). Here, the molar ratio between cholesterol and DSPC (CHOL/DSPC molar ratio), hydration buffer (buffer A, B, C, and D) and the drug to lipid mass ratio (D/L mass ratio) were optimized for preparation. Firstly, lipid mixtures of various CHOL/DSPC molar ratio (ranging from 95/5 to 45/55) were respectively dissolved in chloroform at a total lipid concentration range of 10-30 mg/mL. Next, solvent was removed using rotary evaporation at 25-57° C. with rotation at 400-500 rpm. The resulting well-formed lipid thin film was hydrated with equal volume of buffer solution under sonication to obtain a homogeneous milky suspension. For active drug loading, the suspension was initially adjusted to pH 8-9 using sodium carbonate solution (300 mg/mL). Then various amounts of clonidine hydrochloride was added respectively to each suspension so that the drug to lipid mass ratio was maintained in the range of 1:4 to 1:10. The resulting mixture was then incubated for 1 h at 37° C. Active drug loading was terminated by quenching the mixture on ice for 2 min, and finally MLVs were obtained without the use of extrusion or dialysis procedures.

The encapsulation efficiency (EE, %) of each MLV sample was analyzed by UPLC, and calculated using Equation (3) as follows:

$$EE ( \% ) = \frac{{M}_{0} - {M}_{free}}{{M}_{0}} \times 100 \% \qquad ( 3 )$$

wherein M_{free} refers to the amount of un-encapsulated drug in MLV suspension, and M₀ is the amount of drug originally added to the suspension. Each sample was centrifuged at 20,000 g for 8 min and the supernatant contained un-encapsulated drug was analyzed by UPLC.

For the long-term stability study, freshly prepared MLVs were sealed and stored at 4° C. for further analysis. The changes of total drug amount, i.e. the retention rate of clonidine (RR, %) as well as the encapsulation efficiency (EE, %) of MLVs were evaluated at each time interval.

The retention rate of clonidine (RR, %) during storage was calculated using Equation (4) as follows:

$$RR ( \% ) = \frac{{M}_{t}}{{M}_{0}} \times 100 \% \qquad ( 4 )$$

wherein Mₜ refers to the amount of clonidine retained in MLV suspension at each timepoint, and M₀ is the amount of clonidine originally added to the suspension. Aliquot of MLV was first dissolved in methanol then centrifuged. The supernatant was collected to detect the clonidine content retained in MLV during storage.

### Clonidine Content Analysis Using Ultra Performance Liquid Chromatography (UPLC)

An ACQUITY UPLCH-Class System (Waters, Milford, MA) coupled online to a photodiode array (PDA) detector (wavelength 208 nm) and an evaporative light-scattering detector (ELSD) was used to analyze clonidine concentration of each MLV sample. The samples were separated on a Waters Acquity BEH-C18 column (particle size: 1.7 μm, inner diameter: 2.1 mm, length: 100 mm column) at a flow rate of 0.5 mL/min. The mobile phase was comprised of Solvent A (0.1% v/v aqueous trifluoroacetate) and Solvent B (0.1% v/v trifluoroacetate/acetonitrile). The following gradient was applied: 0 min: A/B (95/5), 3.5 min: A/B (50/50), 3.8 min: A/B (0/100), 4.8 min: A/B (0/100), 5.3 min: A/B (95/5), 6.5 min: A/B (95/5). The concentration of free clonidine in water phase was determined by integrating the PDA peak at a retention time of 1.6 min. The concentration of clonidine dissolved in methanol was determine by integrating the ELSD peak at a retention time of 1.6 min. Data were analyzed using the Empower 3.0 software (Waters).

### Results

### Example 6: Preparation and Characterization of MLVs

Active drug loading is a fast and efficient way to entrap drugs into the core of liposomal particles, avoiding contact with the external environment. Clonidine hydrochloride is a water soluble drug with a bitter taste. Efficient loading into lipid bilayers can not only cover the unpleasant taste of clonidine, but also improve its stability. Here, DSPC/Cholesterol MLVs were employed for clonidine encapsulation. The following is a discussion of the key factors, i.e. CHOL/DSPC molar ratio, hydration buffer (especially pH), and drug/lipid mass ratio, that affect the drug encapsulation efficiency (EE) during MLV preparation and storage.

TABLE 1
Encapsulation efficiency of MLVs prepared with different
CHOL/DSPC molar ratios and hydration buffers

| | CHOL/ | | Drug/ | |
|---|---|---|---|---|
| | DSPC | | Lipid | |
| Sample | molar | | mass | Encapsulation |
| ID | ratio | Hydration Buffer/pH | ratio | efficiency, % |
| M1RA | 45/55 | Buffer A/pH 1.6 | 1:10 | 99.0 ± 0.0 |
| M2RA | 62/38 | Buffer A/pH 1.6 | 1:10 | 97.0 ± 0.0 |
| M3RA | 71/29 | Buffer A/pH 1.6 | 1:10 | 96.3 ± 2.1 |
| M1RB | 45/55 | Buffer B/pH 3.6 | 1:10 | 88.0 ± 1.4 |
| M2RB | 62/38 | Buffer B/pH 3.6 | 1:10 | 88.7 ± 1.2 |
| M3RB | 71/29 | Buffer B/pH 3.6 | 1:10 | 89.3 ± 2.9 |
| M1RC | 45/55 | Buffer C/pH 5.5 | 1:10 | 40.3 ± 4.9 |
| M2RC | 62/38 | Buffer C/pH 5.5 | 1:10 | 55.3 ± 2.5 |
| M3RC | 71/29 | Buffer C/pH 5.5 | 1:10 | 64.7 ± 4.0 |

Table 1 shows the EE of MLVs prepared under the same drug to lipid mass ratio (1:10). As shown in Table 1, compared to CHOL/DSPC molar ratio, hydration buffer and its pH value have a greater impact on drug loading efficiency. Hydration buffer with lower pH value facilitates higher EE. For example, lipid thin films with varying CHOL/DSPC ratios all showed >95% EE using hydration buffer A (pH 1.6). Meanwhile, the lipid composition showed stronger entrapping effect on active drug loading. MLVs with higher CHOL/DSPC molar ratio exhibited higher EE when the pH was increased to 5.5 (buffer C).

TABLE 2
Encapsulation efficiency of MLVs prepared with different
drug/lipid mass ratios and CHOL/DSPC molar ratios

| Drug/Lipid | CHOL/DSPC | | Encapsulation |
|---|---|---|---|
| mass ratio | molar ratio | Hydration Buffer/pH | efficiency, % |
| 1:10 | 47/53 | Buffer D/pH 5.0 | 62.6 |
| | 67/33 | Buffer D/pH 5.0 | 68.7 ± 5.8 |
| | 83/17 | Buffer D/pH 5.0 | 77.9 ± 9.5 |
| | 89/11 | Buffer D/pH 5.0 | 83.0 |
| | 95/5 | Buffer D/pH 5.0 | 56.4 |
| 1:5 | 47/53 | Buffer D/pH 5.0 | 73.2 |
| | 67/33 | Buffer D/pH 5.0 | 71.3 ± 11.8 |
| | 83/17 | Buffer D/pH 5.0 | 73.1 ± 14.9 |
| | 89/11 | Buffer D/pH 5.0 | 73.2 |
| | 95/5 | Buffer D/pH 5.0 | 78.4 |
| 1:4 | 47/53 | Buffer D/pH 5.0 | 59.8 |
| | 67/33 | Buffer D/pH 5.0 | 51.0 |
| | 83/17 | Buffer D/pH 5.0 | 49.4 |
| | 89/11 | Buffer D/pH 5.0 | 38.2 |
| | 95/5 | Buffer D/pH 5.0 | 59.8 |

Considering the effect of drug to lipid ratio on EL of MLVs, as shown in Table 2, lower drug/lipid mass ratio not only results in higher encapsulation efficiency, but also cuts the cost of drug input. Notably, at a drug/lipid ratio of 1:10, the EL ranges from 56-83%, while at a drug/lipid ratio of 1:5, the EL appears more consistent, ranging from 70-80%. However, at a drug/lipid ratio of 1:4, the amount of drug seems to be in excess, causing significant decrease in EL. Moreover, higher CHOL/DSPC molar ratios have a positive effect on EE with the highest EL found in the 2:8 DSPC:Chol ratio.

TABLE 3
Effect of incubation temperature and other preparation
parameters on active drug loading

| | Drug/ | | CHOL/ | |
|---|---|---|---|---|
| | Lipid | | DSPC | |
| Incubation | mass | Hydration | molar | Encapsulation |
| temperature, ° C. | ratio | Buffer/pH | ratio | efficiency, % |
| 25 | 1:3.3 | Buffer B/pH 1.6 | 45/55 | 85 |
| | 1:3.3 | Buffer A/pH 3.6 | 71/29 | 75 |
| 37 | 1:3.3 | Buffer A/pH 1.6 | 71/29 | 97 |
| | 1:3.3 | Buffer B/pH 3.6 | 45/55 | 56 |
| | 1:5 | Buffer B/pH 3.6 | 45/55 | 81 |
| | 1:10 | Buffer B/pH 3.6 | 45/55 | 90 |
| | 1:3.3 | Buffer C/pH 5.5 | 45/55 | 34 |
| 57 | 1:3.3 | Buffer B/pH 3.6 | 45/55 | 94 |
| | 1:5 | Buffer B/pH 3.6 | 45/55 | 78 |
| | 1:10 | Buffer B/pH 3.6 | 45/55 | 99 |

Other parameters such as incubation temperature, drug/lipid mass ratio, etc., during active drug loading were also analyzed. As shown in Table 3, higher incubation temperature facilitates the molecular movement during active loading, therefore resulting in more efficient drug loading. The EL of each sample varies by the complex effect of all preparation parameters.

### Example 7: Stability of MLVs

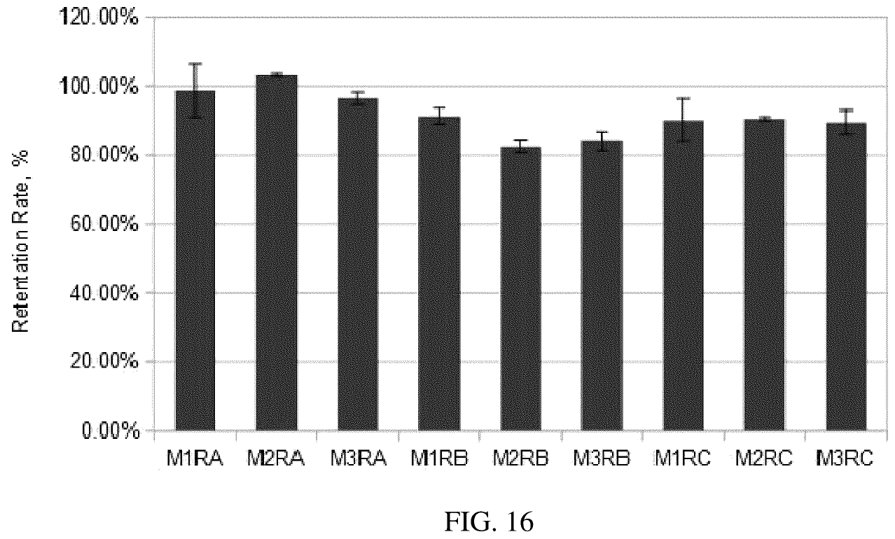

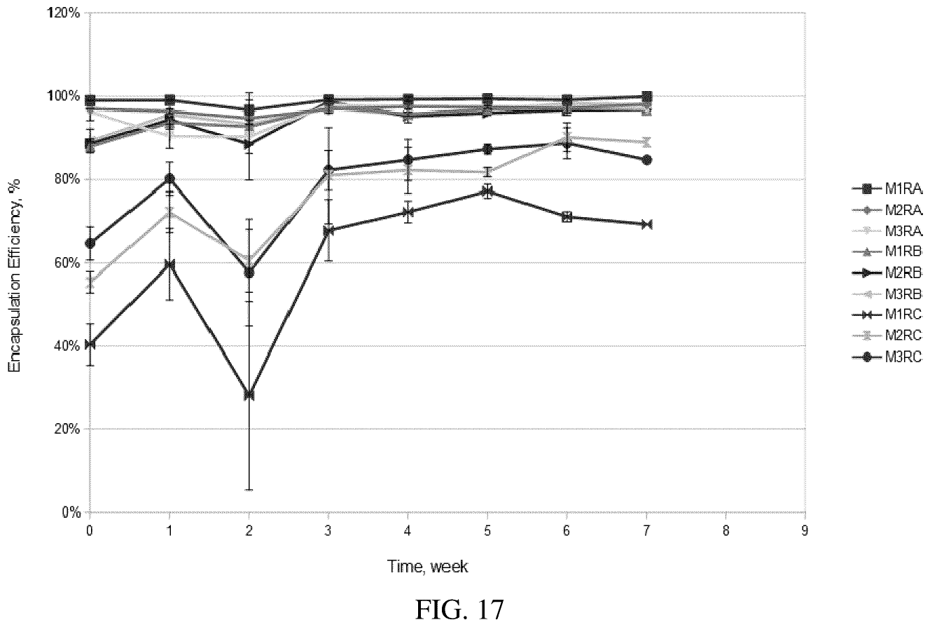

Long-term storage stability of MLVs was evaluated by monitoring the change of total drug amount and EE during 4° C. storage. As shown in FIG. 16, clonidine content in each sample changed little during 7 weeks of storage, with the drug retention rate remaining above 80%. However, the EE of each MLV varied significantly. As shown in FIG. 17, most of the samples showed a trend of decreasing EE during storage. Especially after 4 weeks, samples prepared using buffer A and B showed EE higher than 95%. However, the EE of MLVs prepared using buffer C (pH 5.5) first showed a drastic decrease to <60% in 2 weeks, then gradually rebounded to nearly 80%. These results further indicate that MLVs with higher pH value hindered drug entrapment during active loading.

### REFERENCES

1. British National Formulary for Children|MedicinesComplete. https://about.medicinescomplete.com/publication/british-national-formulary-for-children/. Accessed May 17, 2022.
2. Preventing Medication Errors in Pediatric and Neonatal Patients Defining medication errors.
3. World Health Organization. Development of pediatric medicines: points to consider in pharmaceutical development (Working Document QAS/08.257/Rev.3). 2012; (August).
4. Nunn A J. Making medicines that children can take. Arch Dis Child. 2003; 88(5):369-371. Doi:10.1136/adc.88.5.369
5. Cram A, Breitkreutz J, Desset-Brethes S, Nunn T, Tuleu C. Challenges of developing palatable oral paediatric formulations. Int J Pharm. 2009; 365(1-2):1-3. Doi:10.1016/J.IJPHARM.2008.09.015
6. Tang W L, Tang W H, Chen W C, Diako C, Ross C F, Li S D. Development of a Rapidly Dissolvable Oral Pediatric Formulation for Mefloquine Using Liposomes. Mol Pharm. 2017; 14(6):1969-1979. Doi:10.1021/ACS.MOLPHARMACEUT.7B00077/SUPPL_FILE/MP7B00077_SI_002. AVI
7. Rynes R I. Hydroxychloroquine treatment of rheumatoid arthritis. Am J Med. 1988; 85(4 SUPPL. 1):18-22. Doi:10.1016/0002-9343(88)90357-9
8. Tsakonas E, Joseph L, Esdaile J M, et al. A long-term study of hydroxychloroquine withdrawal on exacerbations in systemic lupus erythematosus. Lupus. 1998; 7(2):80-85. Doi:10.1191/096120398678919778
9. Ruiz-Irastorza G, Egurbide M V., Pijoan J I, et al. Effect of antimalarials on thrombosis and survival in patients with systemic lupus erythematosus. Lupus. 2006; 15(9):577-583. Doi:10.1177/0961203306071872
10. James J A, Kim-Howard X R, Bruner B F, et al. Hydroxychloroquine sulfate treatment is associated with later onset of systemic lupus erythematosus. Lupus. 2007; 16(6):401-409. Doi:10.1177/0961203307078579
11. Sisó A, Ramos-Casals M, Bové A, et al. Previous antimalarial therapy in patients diagnosed with lupus nephritis: influence on outcomes and survival. Lupus. 2008; 17(4):281-288. Doi:10.1177/0961203307086503
12. Pauli E, Joshi H, Vasavada A, Brackett J, Towa L. Evaluation of an Immediate-Release Formulation of Hydroxychloroquine Sulfate With an Interwoven Pediatric Taste-Masking System. J Pharm Sci. 2020; 109(4):1493-1497. Doi:10.1016/J.XPHS.2019.12.014
13. CDC. Medicines for the Prevention of Malaria While Traveling Chloroquine. 2007:1-2.
14. Yu J, Zhou S, Li J, et al. Simple weak-acid derivatives of paclitaxel for remote loading into liposomes and improved therapeutic effects. RSC Adv. 2020; 10(46):27676-27687. Doi:10.1039/D0RA03190A
15. Böttger R, Chao P-H, Fayez N A L, et al. Simultaneous Chromatographic Quantitation of Drug Substance and Excipients in Nanoformulations Using a Combination of Evaporative Light Scattering and Absorbance Detectors. Mol Pharm. May 2022. Doi:10.1021/ACS.MOLPHARMACEUT.2C00021
16. Zhang Y, Huo M, Zhou J, Xie S. PKSolver: An add-in program for pharmacokinetic and pharmacodynamic data analysis in Microsoft Excel. Comput Methods Programs Biomed. 2010; 99(3):306-314. Doi:10.1016/J.CMPB.2010.01.007
17. Rezhdo O, Speciner L, Carrier R L. Lipid-associated Oral Delivery: Mechanisms and Analysis of Oral Absorption Enhancement HHS Public Access. J Control Release. 2016; 240:544-560. Doi:10.1016/j.jconrel.2016.07.050

What is claimed is:

1. A multilamellar vesicle active pharmaceutical ingredient (MLV-API) drug formulation comprising MLV in an aqueous basic solution and an API
wherein the API comprises a weak base drug;
wherein the MLV is loaded with the API; and
wherein pH within the MLV is lower than pH of the aqueous basic solution at the exterior of the MLV.

2. The formulation of claim 1, wherein the drug to lipid ratio of the API loaded MLV is at least about 15% by weight percentage.

3. The formulation of claim 1, wherein the encapsulation efficiency of the API loaded MLV is about 80% or higher.

4. The formulation of claim 1, wherein the pH within the MLV is about 1.0 to about 3.0 and the pH of the aqueous basic solution is about 8.0 to about 10.0.

5. The formulation of claim 1, wherein the API comprises chloroquine (CQ), CQ derivative, clonidine, clonidine derivative, imatinib, propranolol, pilocarpine, meloxicam, aripiprazole, chlorpromazine, R848, imiquimod, mefloquine, primaquine or a pharmaceutically acceptable salt thereof.

6. The formulation of claim 1, wherein the MLV comprises a lipid and cholesterol.

7. The formulation of claim 6, wherein the molar ratio of the cholesterol and the lipid is from about 1:0.2 to about 1:2.2.

8. The formulation of claim 7, wherein the lipid comprises DSPC, DOPC, DOPS, DPPS, DSPS, DOPE, phospholipids, lysolipids, lysophospholipids, sphingolipids, pegylated lipids, phosphatidylserines, phosphatidylglycerols, phosphatidylinositols (not limited to a specific sugar), or a combination thereof.

9. The formulation of claim 1 further comprising cryoprotectants.

10. The formulation of claim 9, wherein the cryoprotectant is at a concentration of about 15% to about 45%.

11. The formulation of claim 1 further comprising one or more compounds other than the API residing in the aqueous basic solution but not encapsulated in the MLV so that the oral drug formulation of the present invention can provide combination therapy of two or more drugs.

12. The formulation of claim 11, wherein the one or more compound comprises an adjuvant.

13. The formulation of claim 1, wherein the API loaded MLV has a size of about 50 to about 500 nm in diameter.

14. The formulation of claim 1, wherein the API loaded MLV has a polydispersity index of about 0.1 to about 0.7.

15. The formulation of claim 1, wherein the API loaded MLV has a zeta potential of about −15 to about 15 mV.

16. The formulation of claim 1, wherein the encapsulation efficiency of the MLV-API drug formulation can be maintained at >=about 80% after about 20 days.

17. The formulation of claim 1, wherein the encapsulation efficiency of the MLV-API drug formulation can be maintained at >=about 94% after about 12 days.

18. The formulation of claim 1, wherein the API loaded MLV provides up to about 95% to about 100% retention of the API within the MLV when the API loaded MLV under physiological conditions equivalent to oral cavity of a subject for up to 30 minutes.

19. The formulation of claim 1, wherein up to about 30% to about 40% of the API encapsulated within the MLV of the MLV-API drug formulation can be released from the MLV under physiological conditions equivalent to stomach of a subject for less than about 120 seconds.

20. The formulation of claim 1, wherein up to about 30% to about 55% of the API encapsulated within the MLV of the MLV-API drug formulation can be released from the MLV under physiological conditions equivalent to stomach of a subject for 4 hours.

21. The formulation of claim 1, wherein up to about 15% to about 40% of the API encapsulated within the MLV of the MLV-API drug formulation can be released from the MLV under physiological conditions equivalent to intestine of a subject for 4 hours.

22. The formulation of claim 1, wherein the drug absorption of the API loaded MLV in a subject is about 1.2- to about 3.0-fold higher than the drug absorption of the free-form drug in a subject.

23. A method of preparation of MLV-API drug formulation comprising the steps of
a. forming MLV by hydrating organic components with an acidic solution;
d. adding an aqueous basic solution to the MLV formed in step a to prepare an aqueous basic solution containing the MLV;
e. dissolving an API in water to prepare an API solution;
f. mixing the API solution of step e with the aqueous basic solution containing the MLV of step d to prepare an active loading solution in which a pH gradient is established between inside and outside of the MLV to facilitate active loading of the API;
g. incubating the active loading solution of step f to effect active loading of the API into the MLV to form API loaded MLV
wherein the API comprises a weak base;
wherein the organic components comprise one or more lipids; and
wherein the method of preparation does not use dialysis or extrusion procedures.

24. The method of claim 23, wherein the drug to lipid ratio of the API loaded MLV is at least about 15% by weight percentage.

25. The method of preparation of claim 23, wherein the encapsulation efficiency realized is 90% or greater when the drug to lipid ratio within the active loading solution of step f is about 5 to about 20 by weight percentage.

26. The method of preparation of claim 23, wherein the encapsulation efficiency realized is 90% or greater when the drug to lipid ratio within the active loading solution of step f is about 15 to about 20 weight percentage and when the API is CQ.

27. The method of preparation of claim 23, wherein the encapsulation efficiency realized is 90% or greater when the drug to lipid ratio within the active loading solution of step f is about 5 by weight percentage and when the API is NB.

28. The method of preparation of claim 23, wherein the encapsulation efficiency realized is 90% or greater when the drug to lipid ratio within the active loading solution of step f is about 5 to about 15 by weight percentage and when the API is HCQ.

29. The method of preparation of claim 23, wherein the encapsulation efficiency realized is 90% or greater when the drug to lipid ratio within the active loading solution of step f is about 10 to about 20 by weight percentage and when the API is clonidine hydrochloride.

30. The method of preparation of claim 23, wherein the incubation temperature of step g is between about 50° C. to about 70° C.

31. The method of preparation of claim 23, wherein the organic components of step a are in the form of a thin film.

32. The method of claim 23, wherein the organic components of step a comprises DSPC, DOPC, DOPS, DPPS, DSPS, DOPE, phospholipids, lysolipids, lysophospholipids, sphingolipids, pegylated lipids, phosphatidylserines, phosphatidylglycerols, phosphatidylinositols (not limited to a specific sugar), cholesterol, or a combination thereof.

33. The method of preparation of claim 23, wherein the organic components of step a comprises cholesterol and lipid at a molar ratio of about 1:0.2 to about 1:2.2.

34. The method of preparation of claim 23 further comprising the step b of adding cryoprotectant and freeze drying the MLV formed in step a.

35. The method of preparation of claim 34, wherein the cryoprotectant is selected from sucrose, dextrose, and trehalose, or a combination thereof.

36. The method of preparation of claim 34, wherein the concentration of cryoprotectant is about 15% to about 45%.

37. The method of preparation of claim 34, further comprising the step c of storing the API loaded MLV at room temperature for up to 8 weeks after the freeze drying step b and before steps d, e, f and g are performed wherein encapsulation efficiency of greater than 90% can be achieved when steps d, e, f and g are performed after step c.

38. The method of preparation of claim 23, wherein the acid solution has pH of about 1.0 to about 3.0.

39. The method of preparation of claim 23, wherein the API comprises chloroquine (CQ), CQ derivative, clonidine, clonidine derivative, imatinib, propranolol, pilocarpine, meloxicam, aripiprazole, chlorpromazine, R848, imiquimod, mefloquine and primaquine or a pharmaceutically acceptable salt thereof.

40. The method of preparation of claim 23, wherein the aqueous basic solution comprises sodium carbonate, potassium carbonate, calcium carbonate, magnesium carbonate, sodium bicarbonate, potassium bicarbonate, calcium bicarbonate, magnesium bicarbonate, sodium hydroxide, potassium hydroxide, calcium hydroxide, magnesium hydroxide, ammonia, or a combination thereof.

41. The method of preparation of claim 23, wherein the MLV-API drug formulation comprises the formulation of claim 1.

42. A method of treatment of rheumatoid arthritis, lupus erythematosus or malaria, high blood pressure, ADHD, drug withdrawal, menopausal flushing, diarrhea, spasticity, and pain conditions comprising orally administering the MLV drug formulation of claim 1 to a subject.

43. The method of claim 42 wherein the subject is a child.

44. A MLV-API drug formulation preparation kit comprising an API, an aqueous basic solution and lyophilized MLV
wherein the API comprises a weak base drug in powder form or any form capable of being ground into powder form;
wherein the three components are stored separately; and
wherein mixing the three components results in active loading of the API into the MLV without requiring any additional ingredients and without extrusion and dialysis procedures to form an MLV-API drug formulation.

45. The kit of claim 44, wherein the MLV-API drug formulation comprises the composition of claim 1.

46. The kit of claim 44 wherein the encapsulation efficiency of the MLV-API drug formulation prepared using the kit of claim 44 can be maintained at >80% up to about 34 days at storage temperature of about 4° C.

47. The kit of claim 44, wherein the encapsulation efficiency of the MLV-API drug formulation prepared using the kit of claim 44 can be maintained at >94% up to about 24 days at storage temperature of at about 4° C.

48. The kit of claim 44, wherein the MLV-API formulation prepared using the kit of claim 44 has encapsulation efficiency of at least 90% after the lyophilized MLV of the kit of claim 45 is stored at room temperature of between about 15° C. to about 30° C. at relative humidity of between about 20% to about 80% for at least 8 weeks.

49. The kit of claim 48, wherein the MLV-API formulation prepared using the kit of claim 44 maintains encapsulation efficiency of at least 90% for about 2 weeks when stored at 4° C.

50. The kit of claim 44, wherein the encapsulation efficiency of the MLV-API drug formulation prepared using the kit of claim 44 is about 90%, about 95%, about 96%, about 97%, about 98% or about 99%.

51. The kit of claim 44, wherein the drug to lipid ratio of the API loaded MLV is at least about 15% by weight percentage.

52. The kit of claim 44, where in the API comprises chloroquine (CQ), CQ derivative, clonidine, clonidine derivative, imatinib, propranolol, pilocarpine, meloxicam, aripiprazole, chlorpromazine, R848, imiquimod, mefloquine and primaquine or a pharmaceutically acceptable salt thereof.

53. The kit of claim 44, wherein the aqueous basic solution comprises sodium carbonate, potassium carbonate, calcium carbonate, magnesium carbonate, sodium bicarbonate, potassium bicarbonate, calcium bicarbonate, magnesium bicarbonate, sodium hydroxide, potassium hydroxide, calcium hydroxide, magnesium hydroxide, ammonia, or a combination thereof.

54. The kit of claim 44, wherein the lyophilized MLV comprises DSPC, DOPC, DOPS, DPPS, DSPS, DOPE, phospholipids, lysolipids, lysophospholipids, sphingolipids, pegylated lipids, phosphatidylserines, phosphatidylglycerols, phosphatidylinositols (not limited to a specific sugar), cholesterol, or a combination thereof.

55. The composition of claim 44, wherein the pH within the MLV is about 1.0 to about 3.0 and the pH of the aqueous basic solution is about 8 to about 10.

56. The kit of claim 44, wherein the molar ratio of cholesterol and DSPC is from about 1:0.2 to about 1:2.2.
